(19) United States
(12) Patent Application Publication
(10) Pub. No.: US 2006/0154990 A1
(43) Pub. Date: Jul. 13, 2006

(54) Use of MEK inhibitors in treating abnormal cell growth

(75) Inventors: Mark Bradley Meyer, Jackson, MI (US); Samuel Eugene DePrimo, San Diego, CA (US); Judith Ann Leopold, Ann Arbor, MI (US); Seth Edward Sadis, Ann Arbor, MI (US); Matthew Adrian Spear, Encinitas, CA (US); Weiwei Tan, San Diego, CA (US); Lloyd Richard Whitfield, Saline, MI (US)

Correspondence Address: AGOURON PHARMACEUTICALS, INC., 10777 SCIENCE CENTER DRIVE, SAN DIEGO, CA, 92121, US

(73) Assignee: AGOURON PHARMACEUTICALS, INC.

(21) Appl. No.: 11/298,084

(22) Filed: Dec. 8, 2005

Related U.S. Application Data
(60) Provisional application No. 60/635,149, filed on Dec. 10, 2004.
(60) Provisional application No. 60/648,972, filed on Jan. 31, 2005.
(60) Provisional application No. 60/680,854, filed on May 12, 2005.
(60) Provisional application No. 60/708,311, filed on Aug. 15, 2005.

(51) Int. Cl. A61K 31/165

(52) U.S. Cl. 514/617

(57) ABSTRACT

This invention relates to use of the compound N-[(R)-2,3-dihydroxy-propoxy]-3,4-difluoro-2-(2-fluoro-4-iodo-phenylamino)-benzamide for treating abnormal cell growth in mammals. In particular, the invention provides dosage regimes for administration of N-[(R)-2,3-dihydroxy-propoxy]-3,4-difluoro-2-(2-fluoro-4-iodo-phenylamino)-benzamide to mammals suffering from cancer.

This application claims the benefit of U.S. Provisional Application No. 60/635,149 filed Dec. 10, 2004; U.S. Provisional Application No. 60/648,972 filed Jan. 31, 2005; U.S. Provisional Application No. 60/680,854 filed May 12, 2005 and U.S. Provisional Application No. 60/708,311 filed Aug. 15, 2005, the contents of which are hereby incorporated by reference in their entireties.

### FIELD OF THE INVENTION

This invention relates to use of MEK inhibitors for treating abnormal cell growth in mammals. In particular, the invention provides dosage regimes for administration of MEK inhibitors to mammals suffering from cancer.

### BACKGROUND OF THE INVENTION

The compound N-[(R)-2,3-dihydroxy-propoxy]-3,4-difluoro-2-(2-fluoro-4-iodo-phenylamino)-benzamide represented by formula 1 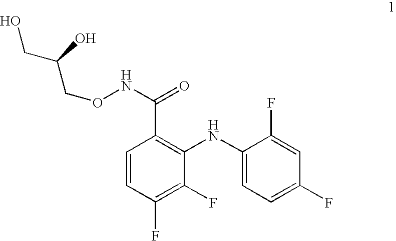
is a highly specific non-ATP-competitive inhibitor of MEK1 and MEK2. The compound of formula 1 (Compound 1) is also known as the compound PD 0325901. Compound 1 is disclosed in WO 02/06213; WO 04/045617; EP 1262176; U.S. patent application Ser. No. 10/144,315 (U.S. Patent Application Publication No. 2003/0055095); U.S. patent application Ser. No. 10/333,399 (U.S. Patent Application Publication No. 2004/0054172); U.S. patent application Ser. No. 10/713,337 (U.S. Patent Application Publication No. 2004/0147478); U.S. Patent Application Publication No. 10/969,681 (U.S. Patent Application Publication No. 2005/0085550); and U.S. patent application Ser. No. 60/690,620, the disclosures of which are incorporated herein by reference in their entireties.

Numerous mitogen-activated protein kinase (MAPK) signaling cascades are involved in controlling cellular processes including proliferation, differentiation, apoptosis, and stress responses. Each MAPK module consists of 3 cytoplasmic kinases: a mitogen-activated protein kinase (MAPK), a mitogen-activated protein kinase kinase (MAPKK), and a mitogen-activated protein kinase kinase kinase (MAPKKK). The RAF-MEK-ERK pathway mediates proliferative and anti-apoptotic signaling from growth factors and oncogenic factors such as Ras and Raf mutant phenotypes that promote tumor growth, progression, and metastasis. Activation of the RAF-MEK-ERK cascade has been demonstrated to be both necessary and sufficient for cell transformation. Mansour et al. “Transformation of mammalian cells by constitutively active MAP kinase.” Science, 1994, v. 265, pp. 966-970; Cowley et al. “Activation of MAP kinase kinase is necessary and sufficient for PC12 differentiation and for transformation of NIH 3T3 cells.” Cell, 1994, v. 77, pp. 841-852; Brunet et al. “Constitutively active mutants of MAP kinase kinase (MEK1) induce growth factor-relaxation and oncogenicity when expressed in fibroblasts.”Oncogene, 1994, v. 9, pp. 3379-3387. By virtue of its central role in mediating the transmission of growth-promoting signals from multiple growth factor receptors, the Ras-MAP kinase cascade provides molecular targets with potentially broad therapeutic applications in oncology. MEK occupies a strategic downstream position in this intracellular signaling cascade catalyzing the phosphorylation of its MAP kinase substrates, ERK1 and ERK2. Anderson et al. “Requirement for integration of signals from two distinct phosphorylation pathways for activation of MAP kinase.” Nature 1990, v.343, pp. 651-653. In the ERK pathway, MAPKK corresponds with MEK (MAP kinase ERK Kinase) and the MAPK corresponds with ERK Lixtracellular Regulated Kinase). No substrates for MEK have been identified other than ERK1 and ERK2. Seger et al. “Purification and characterization of mitogen-activated protein kinase activator(s) from epidermal growth factor-stimulated A431 cells.” J. Biol. Chem., 1992, v. 267, pp. 14373-14381. This tight selectivity in addition to the unique ability to act as a dual-specificity kinase is consistent with MEK's central role in integration of signals into the MAPK pathway. While MEK has not been identified as an oncogene product, MEK is the focal point of many signal transduction mitogenic pathways activated by proven oncogenes. Constitutive action of MAPKs has been reported in >30% of primary tumor cell lines including cell lines derived from colon, lung, breast, pancreas, ovary, and kidney. Hoshino et al. “Constitutive activation of the 41-/43-kDa mitogen-activated protein kinase signaling pathway in human tumors.” Oncogene, 1999, v. 18, pp.813-822. Higher concentrations of active MAPK/ERK (pMAPK/pERK) have been detected in tumor tissue as compared to normal adjacent tissue. Sivaraman et al. “Hyperexpression of mitogen-activated protein kinase in human breast cancer.” J. Clin. Invest., 1997, v. 99, pp. 1478-1483.

Cancer remains a disease with high unmet medical need. Cytotoxic chemotherapy remains the mainstay of systemic therapy for the majority of cancers, particularly late-stage disease. Therefore, cytotoxic agents designed to target steps in molecular pathways unique to, or over-expressed in tumors such as MEK of the RAF-MEK-ERK signaling pathway would fulfill a critical need for cancer patients.

### SUMMARY OF THE INVENTION

In one embodiment, the present invention provides a dosage form for administration to a mammal suffering from cancer, the dosage form comprising N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to reduce phosphorylation of cancer ERK by at least 50%.

In another embodiment, the invention provides a dosage form for administration to a mammal suffering from cancer, the dosage form comprising N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to reduce phosphorylation of cancer ERK by at least 90%.

In another embodiment, the invention provides a dosage form for administration to a mammal suffering from cancer, the dosage form comprising N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to suppress cancer Ki67 by at least 30%.

In another embodiment, the invention provides a dosage form for administration to a mammal, the dosage form comprising N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to reduce phosphorylation of cancer ERK by at least 50% or suppress cancer Ki67 by at least 30% and provide a steady-state average plasma concentration value of at least 7 ng/mL of N-[(R)-2,3-dihydroxy-propoxy]-3,4-difluoro-2-(2-fluoro-4-iodo-phenylamino)-benzamide or active metabolites thereof, for at least 24 hours after administration to the mammal.

In another embodiment, the invention provides an oral dosage form for administration to a mammal, the dosage form comprising N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to reduce phosphorylation of cancer ERK by at least 50% or suppress cancer Ki67 by at least 30% and provide a steady-state average plasma concentration value of at least 7 ng/mL of N-[(R)-2,3-dihydroxy-propoxy]-3,4-difluoro-2-(2-fluoro-4-iodo-phenylamino)-benzamide or active metabolites thereof, for at least 24 hours after administration to the mammal. In another embodiment, the invention provides an oral dosage form, wherein the dosage form is a tablet or a capsule.

In another embodiment, the invention provides a dosage form for administration to a mammal, the dosage form comprising N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount of no more than 30 mg.

In another embodiment, the invention provides a dosage form for administration to a mammal, the dosage form comprising N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount of from 1 to 20 mg.

In another embodiment, the invention provides a method of treating cancer in a mammal, the method comprising administering to the mammal N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to reduce phosphorylation of cancer ERK by at least 50%.

In another embodiment, the invention provides a method of treating cancer in a mammal, the method comprising administering to the mammal N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to reduce phosphorylation of cancer ERK by at least 90%.

In another embodiment, the invention provides a method of treating cancer in a mammal, the method comprising administering to the mammal N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to suppress cancer Ki67 by at least 30%.

In another embodiment, the invention provides a method of treating cancer in a mammal, the method comprising administering to the mammal N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to reduce phosphorylation of cancer ERK by at least 50% or suppress a cancer Ki67 by at least 30% and provide a steady-state average plasma concentration value of at least 7 ng/mL of N-[(R)-2,3-dihydroxy-propoxy]-3,4-difluoro-2-(2-fluoro-4-iodo-phenylamino)-benzamide or active metabolites thereof, for at least 24 hours after administration to the mammal.

In another embodiment, the invention provides a method of treating cancer in a mammal, the method comprising administering to the mammal N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to reduce phosphorylation of cancer ERK by at least 50% or suppress cancer Ki67 by at least 30% wherein N-[(R)-2,3-dihydroxy-propoxy]-3,4-difluoro-2-(2-fluoro-4-iodo-phenylamino)-benzamide is administered at a dosage frequency of at least once per day.

In another embodiment, the invention provides a method of treating cancer in a mammal, the method comprising administering to the mammal N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to reduce phosphorylation of cancer ERK by at least 50% or suppress cancer Ki67 by at least 30% wherein N-[(R)-2,3-dihydroxy-propoxy]-3,4-difluoro-2-(2-fluoro-4-iodo-phenylamino)-benzamide is administered at a dosage frequency of at least twice per day.

Definitions and Abbreviations of Terms

“Abnormal cell growth”, as used herein, unless otherwise indicated, refers to cell growth that is independent of normal regulatory mechanisms (e.g., loss of contact inhibition).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The phrase “pharmaceutically acceptable salt(s)”, as used herein, unless otherwise indicated, includes salts of acidic or basic groups, which may be present in a compound. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

The invention also includes isotopically-labeled compounds, which are identical to this recited in Formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention and pharmaceutically acceptable salts of said compounds, which contain the aforementioned isotopes and/or other isotopes of other atoms, are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. An isotopically labeled Compound 1 of this invention can generally be prepared by carrying out the procedures described for the non-labeled compound, substituting a readily available isotopically labeled reagent for a non-isotopically labeled reagent.
AE adverse event
AR accumulation ratio
AUC area under the plasma concentration-time curve
AUC₍₀₋₂₄₎ area under the plasma concentration-time curve from 0 to 24 hours
AUC₍₀₋ₜₗₐₛₜ₎ area under the plasma concentration-time curve from time 0 to the last recorded observation
BID twice per day (bis in die)
C₀ initial concentration
CL clearance
Cₘₐₓ maximum plasma concentration
Css, avg steady-state average plasma concentrations
C_{through} pre-dose concentration
CV % coefficient of variation in percentile
DLT dose-limiting toxicities
EC₅₀ concentration producing 50% of maximum effect
ECG electrocardiogram
ERK Extracellular Regulated Kinase
pERK phosphorylated ERK
IC₅₀ 50% inhibitory concentration
IGF insulin-like growth factor
IGF-1R insulin-like growth factor receptor, Type 1
IP intraperitoneal
IV intravenous
MTD maximum tolerated dose
NAD nicotinamide adenine dinucleotide
PD pharmacodynamic
PK pharmacokinetic
PO orally
QD once per day (quaque die)
Tₘₐₓ time of occurrence of Cₘₐₓ

Response Criteria

The following RECIST criteria was the primary method utilized in this study for the assessment and reporting of tumor response data.

Complete Response (CR): Disappearance of all target and nontarget lesions and no appearance of new lesions indicates complete response. Each of these must be documented on 2 occasions separated by at least 4 weeks.

Partial Response (PR): At least a 30% decrease in the sum of the LDs of target lesions (taking as reference the baseline sum), without progression of nontarget lesions and no appearance of new lesions indicates partial response. Each of these must be documented on 2 occasions separated by at least 4 weeks.

Stable Disease (SD): Measurements demonstrating neither sufficient shrinkage to qualify for PR, nor sufficient increase to qualify as PD during the first 8 weeks after the start of treatment, taking as reference the smallest sum LD since the treatment started. During this time, nontarget lesions may persist provided there is no unequivocal progression in these lesions. This must be confirmed on a 2^{nd} occasion separated by at least 4 weeks.

Progressive Disease (PD): ≧20% increase in the sum of the LD of target lesions taking as references the smallest sum LD recorded since the treatment started, unequivocal progression of existing nontarget lesions, or the appearance of 1 or more new lesions. The occurrence of a pleural effusion or ascites is also considered PD if substantiated by cytologic investigation and not previously documented. Pathologic fracture or collapse of bone is not necessarily evidence of disease progression; however, new bone lesions not previously documented are considered PD.

### DETAILED DESCRIPTION OF THE INVENTION

I. Pharmaceutical Formulations of N-[(R)-2,3-dihydroxy-propoxy]-3,4-difluoro-2-(2-fluoro-4-iodo-phenylamino)-benzamide (Compound 1)

Compound 1 can be prepared as described in WO 02/06213; U.S. patent application Ser. No. 10/333,399 and U.S. patent application Ser. No. 60/690,620. U.S. patent application Ser. No. 10/969,681 discloses a novel polymorphic form IV of Compound 1.

Administration of Compound 1 can be effected by any method that enables delivery of the compound to the site of action. These methods include oral routes, intraduodenal routes, parenteral injection (including intravenous, subcutaneous, intramuscular, intravascular or infusion), topical, and rectal administration.

The compound may, for example, be provided in a form suitable for oral administration as a tablet, capsule, pill, powder, sustained release formulation, solution, suspension, for parenteral injection as a sterile solution, suspension or emulsion, for topical administration as an ointment or cream or for rectal administration as a suppository.

The compound may be in unit dosage forms suitable for single administration of precise dosages. Preferably, dosage forms include a conventional pharmaceutical carrier or excipient and Compound 1 as an active ingredient. In addition, dosage forms may include other medicinal or pharmaceutical agents, carriers, adjuvants, etc.

Exemplary parenteral administration forms include solutions or suspensions in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical composition may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials therefor include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

In preferred embodiments of the dosage forms of the invention, the dosage form is an oral dosage form, more preferably, a tablet or a capsule.

In preferred embodiments of the dosage forms of the invention, the dosage form is an oral dosage form, more preferably, a tablet or a capsule.

In preferred embodiments of the methods of the invention, Compound 1 is administered orally, such as, for example, using an oral dosage form as described herein.

The methods include administering Compound 1 using any desired dosage regimen. In one specific embodiment, the compound is administered once per day (quaque die, or QD), preferably twice per day (bis in die, or BID), although more or less frequent administration is within the scope of the invention. For example, the dose regimen may be twice daily (BID) dosing for 21 days repeated every 28 days or Compound 1 can be administered continuously without interruption between 21-day cycles. The compound can be administered to the mammal, including a human, in a fasted state (for example, no food or beverage within 2 hours before and after administration) or, preferably, with food.

Methods of preparing various dosage forms with a specific amount of Compound 1 are known, or will be apparent, to those skilled in this art. For examples, see Remington's Pharmaceutical Sciences, Mack Publishing Company, Easter, Pa., 15th Edition (1975).

According to the present invention, a dosage form for administration to a mammal suffering from cancer comprises N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to reduce phosphorylation of cancer ERK by at least 50%, preferably, at least 90%. The phosphorylation status of ERK can be assessed, for example, through antibody-based detection methods. A phosphorylation site-specific antibody, such as, an antibody which reacts specifically with the dually phosphorylated form of MAPK (both the ERK-1 and ERK-2 forms) can be used as described in Example 10.

In another embodiment, a dosage form for administration to a mammal suffering from cancer comprises N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to suppress the proliferation marker Ki67 by at least 30%. The amount of Ki67 can be detected, for example, by immunohistochemistry as described in Example 11.

II. Use of Pharmaceutical Compositions of the Present Invention

This invention also relates to a method for the treatment of abnormal cell growth in a mammal, including a human, comprising administering to said mammal an amount of Compound 1, as defined above, or a pharmaceutically acceptable salt or solvate thereof, that is effective in treating abnormal cell growth.

In one embodiment of this method, the abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of Compound 1, or a pharmaceutically acceptable salt or solvate thereof, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a pharmaceutical composition for the treatment of abnormal cell growth in a mammal, including a human, comprising an amount of Compound 1, as defined above, or a pharmaceutically acceptable salt or solvate thereof, that is effective in treating abnormal cell growth, and a pharmaceutically acceptable carrier. In one embodiment of said composition, said abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers. In another embodiment of said pharmaceutical composition, said abnormal cell growth is a benign proliferative disease, including, but not limited to, psoriasis, benign prostatic hypertrophy or resbnosis.

The invention also relates to a pharmaceutical composition for the treatment of abnormal cell growth in a mammal, including a human, which comprises an amount of Compound 1, as defined above, or a pharmaceutically acceptable salt or solvate thereof, that is effective in treating abnormal cell growth in combination with a pharmaceutically acceptable carrier and an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

The invention also relates to a method for the treatment of a hyperproliferative disorder in a mammal which comprises administering to said mammal a therapeutically effective amount of Compound 1, or a pharmaceutically acceptable salt or hydrate thereof, in combination with an anti-tumor agent selected from the group consisting of antiproliferative agents, kinase inhibitors, angiogenesis inhibitors, growth factor inhibitors, cox-I inhibitors, cox-II inhibitors, mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, statins, and anti-androgens.

One with the ordinary skill in the art can appreciate that Compound 1 may be administered in many possible regimens, for example, continuously, daily, adjusting the dose if any additional toxicity is encountered in the combination.

In one embodiment of the present invention the anti-tumor agent used in conjunction with Compound 1 and pharmaceutical compositions described herein is an anti-angiogenesis agent, kinase inhibitor, pan kinase inhibitor or growth factor inhibitor.

Preferred pan kinase inhibitors include SU-11248, described in U.S. Pat. No. 6,573,293 (Pfizer, Inc, NY, USA).

Anti-angiogenesis agents, include but are not limited to the following agents, such as EGF inhibitor, EGFR inhibitors, VEGF inhibitors, VEGFR inhibitors, TIE2 inhibitors, IGF1R inhibitors, COX-II (cyclooxygenase II) inhibitors, MMP-2 (matrix-metalloproteinase 2) inhibitors, and MMP-9 (matrix-metalloproteinase 9) inhibitors.

Preferred VEGF inhibitors, include for example, Avastin (bevacizumab), an anti-VEGF monoclonal antibody of Genentech, Inc. of South San Francisco, Calif.

Additional VEGF inhibitors include CP-547,632 (Pfizer Inc., NY, USA), AG13736 (Pfizer Inc.), ZD-6474 (AstraZeneca), AEE788 (Novartis), AZD-2171), VEGF Trap (Regeneron,/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof. VEGF inhibitors useful in the practice of the present invention are disclosed in U.S. Pat. Nos. 6,534,524 and 6,235,764, both of which are incorporated in their entirety for all purposed.

Particularly preferred VEGF inhibitors include CP-547,632, AG13736, Vatalanib, Macugen and combinations thereof.

Additional VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published December 2, 1999), U.S. Pat. No. 6, 534,524 (discloses AG13736), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued March 23, 1999), U.S. Pat. No. 5,792,783 (issued Aug. 11, 1998), U.S. Pat. No. US 6,653,308 (issued Nov. 25, 2003), WO 99/10349 (published Mar. 4,1999), WO 97/32856 (published Sep. 12,1997), WO 97/22596 (published June 26,1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following U.S. Pat. application Nos. 09/221946 (filed Dec. 28, 1998); 09/454058 (filed Dec. 2, 1999); 09/501163 (filed Feb. 9, 2000); 09/539930 (filed Mar. 31, 2000); 09/202796 (filed May 22, 1997); 09/384339 (filed Aug. 26, 1999); and 09/383755 (filed Aug. 26, 1999); and the compounds disclosed and claimed in the following United States provisional patent application Nos.: 60/168207 (filed Nov. 30, 1999); 60/170119 (filed Dec. 10, 1999); 60/177718 (filed Jan. 21, 2000); 60/168217 (filed Nov. 30, 1999), and 60/200834 (filed May 1, 2000). Each of the foregoing patent applications and provisional patent applications is herein incorporated by reference in their entirety.

PDGRr inhibitors include but not limited to those disclosed international patent application publication number WO01/40217, Published Jul. 7, 2001 and international patent application publication number WO2004/020431, Published Mar. 11, 2004, the contents of which are incorporated in their entirety for all purposes.

Preferred PDGFr inhibitors include Pfizer's CP-673,451 and CP-868,596 and its pharmaceutically acceptable salts.

Preferred GARF inhibitors include Pfizers AG-2037 (pelitrexol and its pharmaceutically acceptable salts. GARF inhibitors useful in the practice of the present invention are disclosed in U.S. Pat. No. 5,608,082 which is incorporated in its entirety for all purposed.

Examples of useful COX-II inhibitors which can be used in conjunction with the compound of formula land pharmaceutical compositions described herein include CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), parecoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additonally, COX-II inhibitors are disclosed in U.S. patent application Ser. Nos. 10/801,446 and 10/801,429, the contents of which are incorporated in their entirety for all purposes.

In one preferred embodiment the anti-tumor agent is celecoxib as disclosed in U.S. Pat. No. 5,466,823, the contents of which are incorporated by reference in its entirety for all purposes. The structure for Celecoxib is shown below:

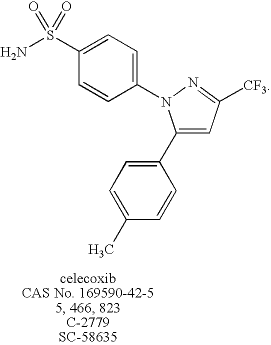

In one preferred embodiment the anti-tumor agent is valecoxib as disclosed in U.S. Pat. No. 5,633,272, the contents of which are incorporated by reference in its entirety for all purposes. The structure for valdecoxib is shown below:

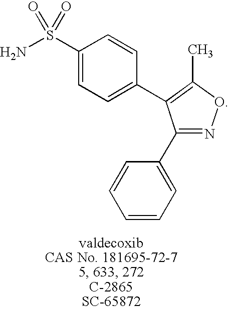

In one preferred embodiment the anti-tumor agent is parecoxib as disclosed in U.S. Pat. No. 5,932,598, the contents of which are incorporated by reference in its entirety for all purposes. The structure for parecoxib is shown below:

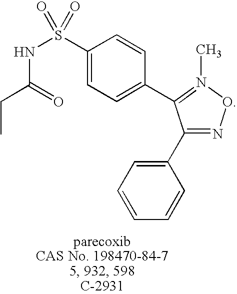

In one preferred embodiment the anti-tumor agent is deracoxib as disclosed in U.S. Pat. No. 5,521,207, the contents of which are incorporated by reference in its entirety for all purposes. The structure for deracoxib is shown below:

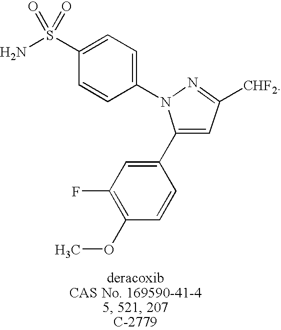

In one preferred embodiment the anti-tumor agent is SD-8381 as disclosed in U.S. Pat. No. 6,034,256, the contents of which are incorporated by reference in its entirety for all purposes. The structure for SD-8381 is shown below:

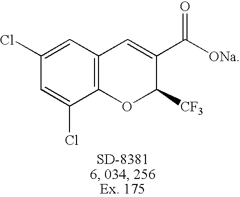

In one preferred embodiment the anti-tumor agent is ABT-963 as disclosed in International Publication Number WO 2002/24719, the contents of which are incorporated by reference in its entirety for all purposes. The structure for ABT-963 is shown below:

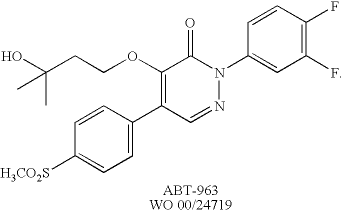

In one preferred embodiment the anti-tumor agent is rofecoxib as shown below:

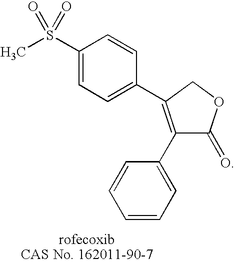

In one preferred embodiment the anti-tumor agent is MK-663 (etoricoxib) as disclosed in International Publication Number WO 1998/03484, the contents of which are incorporated by reference in its entirety for all purposes. The structure for etoricoxib is shown below:

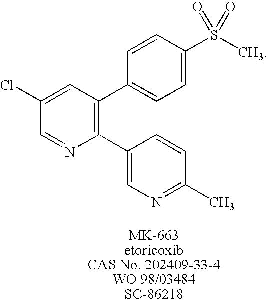

In one preferred embodiment the anti-tumor agent is COX-189 (Lumiracoxib) as disclosed in International Publication Number WO 1999/11605, the contents of which are incorporated by reference in its entirety for all purposes. The structure for Lumiracoxib is shown below:

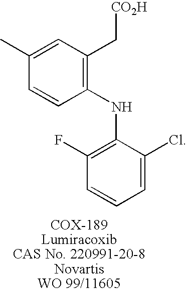

In one preferred embodiment the anti-tumor agent is BMS-347070 as disclosed in U.S. Pat. No. 6,180,651, the contents of which are incorporated by reference in its entirety for all purposes. The structure for BMS-347070 is shown below:

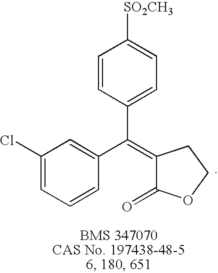

In one preferred embodiment the anti-tumor agent is NS-398 (CAS 123653-11-2). The structure for NS-398 (CAS 123653-11-2) is shown below:

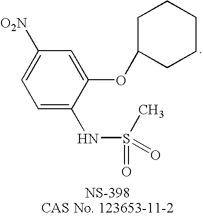

In one preferred embodiment the anti-tumor agent is RS 57067 (CAS 17932-91-3). The structure for RS-57067 (CAS 17932-91-3) is shown below:

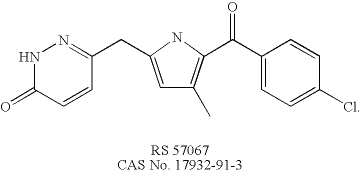

In one preferred embodiment the anti-tumor agent is 4-Methyl-2-(3,4-dimethylphenyl)-1-(4-sulfamoyl-phenyl)-1H-pyrrole. The structure for 4-Methyl-2-(3,4-dimethylphenyl)-1-(4-sulfamoyl-phenyl)-1H-pyrrole is shown below:

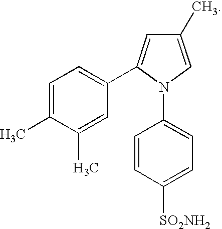

In one preferred embodiment the anti-tumor agent is 2-(4-Ethoxyphenyl)-4-methyl-1-(4-sulfamoylphenyl)-1H-pyrrole. The structure for 2-(4-Ethoxyphenyl)-4-methyl-1-(4-sulfamoylphenyl)-1H-pyrrole is shown below:

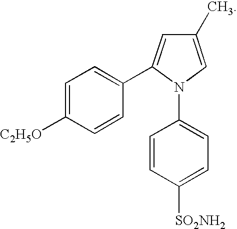

In one preferred embodiment the anti-tumor agent is meloxicam. The structure for meloxicam is shown below:

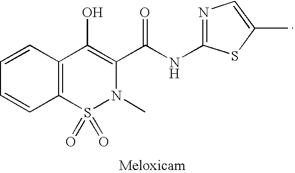

Other useful inhibitors as anti-tumor agents used in conjunction with the compound of formula land pharmaceutical compositions described herein include aspirin, and non-steroidal anti-inflammatory drugs (NSAIDs) which inhibit the enzyme that makes prostaglandins (cyclooxygenase I and II), resulting in lower levels of prostaglandins, include but are not limited to the following, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Preferred COX-I inhibitors include ibuprofen (Motrin), nuprin, naproxen (Aleve), indomethacin (Indocin), nabumetone (Relafen) and combinations thereof.

Targeted agents used in conjunction with the compound of formula land pharmaceutical compositions described herein include EGFr inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof Preferred EGFr inhibitors include Iressa, Erbitux, Tarceva and combinations thereof.

The present invention also relates to anti-tumor agents selected from pan erb receptor inhibitors or ErbB2 receptor inhibitors, such as CP-724,714 (Pfizer, Inc.), C1-1033 (canertinib, Pfizer, Inc.), PF-00299804 (Pfizer, Inc.), Herceptin (trastuzumab, Genentech Inc.), Omitarg (2C4, pertuzumab, Genentech Inc.), TAK-165 (Takeda), GW-572016 (lonafarnib, GlaxoSmithKline), GW-282974 (GlaxoSmithKline), EKB-569 (Wyeth), PKI-166 (Novartis), dHER2 (HER2 Vaccine, Corixa and GlaxoSmithKline), APC8024 (HER2 Vaccine, Dendreon), anti-HER2/neu bispecific antibody (Decof Cancer Center), B7.her2.1gG3 (Agensys), AS HER2 (Research Institute for Rad Biology & Medicine), trifuntional bispecific antibodies (University of Munich) and mAB AR-209 (Aronex Pharmaceuticals Inc) and mAB 2B-1 (Chiron) and combinations thereof. Preferred erb selective anti-tumor agents include Herceptin, TAK-165, CP-724,714, ABX-EGF, HER3 and combinations therebf.

Preferred pan erbb receptor inhibitors include GW572016, Cl-1033, PF-00299804, EKB-569, and Omitarg and combinations thereof.

Additional erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Pat. Nos. 6,465,449, and 6,284,764, and International Application No. WO 2001/98277 each of which are herein incorporated by reference in their entirety.

Additionally, other anti-tumor agents may be selected from the following agents, BAY-43-9006 (Onyx Pharmaceuticals Inc.), Genasense (augmerosen, Genta), Panitumumab (Abgenix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aetema), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antisoma), Thymitaq (Eximias), Temodar (temozolomide, Schering Plough) and Revilimd (Celegene) and combinations thereof.

Other anti-tumor agents may be selected from the following agents, CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE), ABI-007 (paclitaxel), Evista (raloxifene), Atamestane (Biomed-777), Xyotax (polyglutamate paclitaxel), Targetin (bexarotine) and combinations thereof.

Additionally, other anti-tumor agents may be selected from the following agents, Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpirnase), BEC2 (mitumoab), Xcytrin (motexafin gadolinium) and combinations thereof.

Further anti-tumor agents may selected from the following agents, CeaVac (CEA), NeuTrexin (tnmetresate glucuronate) and combinations thereof.

Additional anti-tumor agents may selected from the following agents, OvaRex (oregovomab), Osidem (IDM-1), and combinations thereof.

Additional anti-tumor agents may selected from the following agents, Advexin (ING 201), Tirazone (tirapazamine), and combinations thereof.

Additional anti-tumor agents may selected from the following agents, RSR13 (efaproxiral), Cotara (1311 chTNT 1/b), NBI-3001 (IL-4) and combinations thereof.

Additional anti-tumor agents may selected from the following agents, Canvaxin, GMK vaccine, PEG Interon A, Taxoprexin (DHA/paciltaxel) and combinations thereof.

Other preferred anti-tumor agents include Array Biopharma's MEK inhibitor ARRY-142886, Bristol Myers' CDK2 inhibitor BMS-387,032, Pfizer's CDK inhibitor PD0332991 and AG-024322 and AstraZeneca's AXD-5438 and combinations thereof.

Additionally, mTOR inhibitors may also be utilized such as Temsirolimus, CCI-779 (Wyeth) and rapamycin derivatives RAD001 (Novartis) and AP-23573 (Ariad), HDAC inhibitors SAHA (Merck Inc./Aton Pharmaceuticals) and combinations thereof.

Additional anti-tumor agents include aurora 2 inhibitor VX-680 (Vertex), Chk1/2 inhibitor XL844 (Exilixis).

The following cytotoxic agents, e.g., one or more selected from the group consisting of epirubicin (Ellence), docetaxel (Taxotere), paclitaxel, Zinecard (dexrazoxane), rituximab (Rituxan) imatinib mesylate (Gleevec), and combinations thereof, may be used in conjunction with the compound of formula 1 and pharmaceutical compositions described herein.

The invention also contemplates the use of the compounds of the present invention together with hormonal therapy, including but not limited to, exemestane (Aromasin, Pfizer Inc.), leuprorelin (Lupron or Leuplin, TAP/Abbott/Takeda), anastrozole (Arimidex, Astrazeneca), gosrelin (Zoladex, AstraZeneca), doxercalciferol, fadrozole, formestane, tamoxifen citrate (tamoxifen, Nolvadex, AstraZeneca), Casodex (AstraZeneca), Abarelix (Praecis), Trelstar, and combinations thereof. For example, aromasin might be delivered in the following schedule: 25 mg po qd continuously. Coombes et al. New England J. Med., 2004, vol. 350, No. 11, pp. 1081-1092.

The invention also relates to hormonal therapy agents such as anti-estrogens including, but not limited to fulvestrant, toremifene, raloxifene, lasofoxifene, letrozole (Femara, Novartis), anti-androgens such as bicalutamide, flutamide, mifepristone, nilutamide, Casodex®(4′-cyano-3-(4-fluorophenylsulphonyl)-2-hydroxy-2-methyl-3′-(trifluoromethyl) propionanilide, bicalutamide) and combinations thereof.

Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

Particularly preferred cytotoxic agents include Camptosar, Erbitux, Iressa, Gleevec, Taxotere and combinations thereof.

The following topoisomerase I inhibitors may be utilized as anti-tumor agents camptothecin, irinotecan HCl (Camptosar), edotecarin, orathecin (Supergen), exatecan (Daiichi), BN-80915 (Roche) and combinations thereof.

Particularly preferred topoisomerase II inhibitors include epirubicin (Ellence).

The compounds of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers.

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, Paraplatin (carboplatin), eptaplatin, lobaplatin, nedaplatin, Eloxatin (oxaliplatin, Sanofi) or satrplatin and combinations thereof.

Particularly preferred alkylating agents include Eloxatin (oxaliplatin).

Antimetabolites include but are not limited to, methotrexate, 6-mercaptopurne riboside, mercaptopurine, 5-fluorouracil (5-FU) alone or in combination with leucovorin, tegafur, UFT, doxifluridine, carmofur, cytarabine, cytarabine ocfosfate, enocitabine, S-1, Alimta (premetrexed disodium, LY231514, MTA), Gemzar (gemcitabine, Eli Lilly), fludarabin, 5-azacitidine, capecitabine, cladribine, clofarabine, decitabine, eflomithine, ethynylcytidine, cytosine arabinoside, hydroxyurea, TS-1, melphalan, nelarabine, nolatrexed, ocfosfate, disodium premetrexed, pentostatin, pelitrexol, raltitrexed, triapine, trimetrexate, vidarabine, vincristine, vinorelbine; or for example, one of the preferred anti-metabolites disclosed in European patent application Ser. No. 239362 such as N-(5-[N-(3,4-dihydro-2-methyl-4-oxoqui nazolin-6-ylmethyl)-N-methylamino]-2-thenoyl)-L-glutamic acid and combinations thereof.

For example, gemcitabine might be delivered in the following schedule: 1000 mg/m², 1 Per week, 3 weeks of every 4 weeks. El-Rayes et al. J. Clin. Oncol., 2003, vol. 21, No. 15, pp. 2920-2925.

Antibiotics include intercalating antibiotics but are not limited to: aclarubicin, actinomycin D, amrubicin, annamycin, adriamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin, zinostatin and combinations thereof.

Plant derived anti-tumor substances include for example those selected from mitotic inhibitors, for example vinblastine, docetaxel (Taxotere), paclitaxel and combinations thereof.

Cytotoxic topoisomerase inhibiting agents include one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, topotecan, and combinations thereof.

Preferred cytotoxic topoisomerase inhibiting agents include one or more agents selected from the group consisting of camptothecin, 10-hydroxycamptothecin, 9-aminocamptothecin, irinotecan HCl (Camptosar), edotecarin, epirubicin (Ellence), etoposide, SN-38, topotecan, and combinations thereof.

Immunologicals include interferons and numerous other immune enhancing agents. Interferons include interferon alpha, interferon alpha-2a, interferon, alpha-2b, interferon beta, interferon gamma-1a, interferon gamma-1b (Actimmune), or interferon gamma-ni and combinations thereof. Other agents include filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab (Y-muHMFG1), Provenge (Dendreon) and combinations thereof.

Biological response modifiers are agents that modify defense mechanisms of living organisms or biological responses, such as survival, growth, or differentiation of tissue cells to direct them to have anti-tumor activity. Such agents include krestin, lentinan, sizofiran, picibanil, ubenimex and combinations thereof.

Other anticancer agents include alitretinoin, ampligen, atrasentan bexarotene, bortezomib. Bosentan, calcitriol, exisulind, finasteride, fotemustine, ibandronic acid, miltefosine, mitoxantrone, I-asparaginase, procarbazine, dacarbazine, hydroxycarbamide, pegaspargase, pentostatin, tazarotne, Telcyta (TLK-286, Telik Inc.), Velcade (bortemazib, Millenium), tretinoin, and combinations thereof.

Other anti-angiogenic compounds include acitretin, fenretinide, thalidomide, zoledronic acid, angiostatin, aplidine, cilengtide, combretastatin A4, endostatin, halofuginone, rebimastat, removab, Revlimid, squalamine, ukrain, Vitaxin and combinations thereof.

Platinum-coordinated compounds include but are not limited to, cisplatin, carboplatin, nedaplatin, oxaliplatin, and combinations thereof.

Camptothecin derivatives include but are not limited to camptothecin, 10-hydroxycamptothecin, 9-aminocamptothecin, irinotecan, SN-38, edotecarin, topotecan and combinations thereof.

Other antitumor agents include mitoxantrone, I-asparaginase, procarbazine, dacarbazine, hydroxycarbamide, pentostatin, tretinoin and combinations thereof.

Anti-tumor agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736; and anti-proliferative agents such as other farnesyl protein transferase inhibitors, for example the farnesyl protein transferase inhibitors. Additional, specific CTLA4 antibodies that can be used in the present invention include those described in U.S. Provisional Application 60/113,647 (filed Dec. 23, 1998), U.S. Pat. No. 6, 682,736 both of which are herein incorporated by reference in their entirety.

In one embodiment of the present invention, protease inhibitors such as nelfinavir (trade name viracept), one of a new class of anti-HIV drugs, may be used in conjunction with Compound 1 and pharmaceutical compositions. Protease inhibitors work by blocking a part of HIV called protease. When protease is blocked, HIV makes copies of itself that can't infect new cells. Protease inhibitors are almost always used in combination with at least two other anti-HIV drugs.

For example, nelfinavir might be delivered in the following schedule: 1250 mg po bid continuously. Regazzi et al. Antimicrobial Agents & Chemotherapy, 2005, vol. 49, No. 2, pp. 643-649.

Specific IGF1 R antibodies that can be used in the present invention include those described in International patent application Ser. No. WO 2002/053596, which is herein incorporated by reference in its entirety.

Specific CD40 antibodies that can be used in the present invention include those described in International patent application Ser. No. WO 2003/040170 which is herein incorporated by reference in its entirety.

Gene therapy agents may also be employed as anti-tumor agents such as TNFerade (GeneVec), which express TNFalpha in response to radiotherapy.

In one embodiment of the present invention statins may be used in conjunction with Compound 1 and pharmaceutical compositions. Statins (HMG-COA reducatase inhibitors) may be selected from the group consisting of Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin and Niacin (Advicor, Kos Pharmaceuticals), derivatives and combinations thereof.

In a preferred embodiment the statin is selected from the group consisting of Atovorstatin and Lovastatin, derivatives and combinations thereof.

Other agents useful as anti-tumor agents include Caduet.

The disclosures of all cited references are incorporated herein by reference in their entirety.

### EXAMPLES

The examples and preparations provided below further illustrate and exemplify the use of Compound 1 for treating abnormal cell growth in mammals including the dosage regimes for administration of Compound 1 to mammals suffering from cancer.

It is to be understood that the scope of the present invention is not limited in any way by the scope of the following examples.

### Exmple 1

### Inhibition of MEK1, MEK2, and Enzyme Specificity

Compound 1 was shown to be a selective inhibitor of MEK1 and MEK2 in vitro. The Ki^{app} of Compound 1 for an activated form of MEK1, MEK1-S218D/S222D, was 1.1±0.2 nM, while the KiaPP of Compound 1 for an activated form of MEK2, MEK2-S222D/S226D was 0.79±0.2 nM. Compound 1 was also tested in a “cascade” assay in which activated B-Raf, unactivated MEK1, and ERK1 were present. In this cascade assay activated B-Raf phosphorylates and activates MEK1, and activated MEKI in turn phosphorylates ERK1. The readout is phosphorylation of ERK1. Inhibitors that bind to either the unactivated or the activated form of MEK1 can inhibit this assay. The Ki^{app} for Compound 1 was 0.90±0.09 nM in the cascade assay. The specificity of Compound 1 was evaluated against a panel of 27 kinases. This panel, which was comprised of tyrosine kinases as well as a multitude of serne/threonine kinases, was completely refractory to inhibition by Compound 1 at a concentration of 10 μM. Therefore, Compound 1 appears to be highly specific for MEK1/2.

### Example 2

### Kinetics of Inhibition of MEK1

Table 1 represents biological and pharmacologic properties of Compound 1 The kinetics of MEK1-S218D/S222D inhibition in vitro by Compound 1 was studied by evaluating the IC₅₀ as a function of substrate concentration. This was done instead of the classic Lineweaver-Burke analysis of evaluating inhibitor mechanism, because the Ki^{app} of Compound 1 (˜1 nM) is lower than the enzyme concentration used in vitro (˜10 nM). Copeland R A. In Enzymes: A Practical Introduction to Structure, Mechanism and Data Analysis. New York: Wiley-VCH Press; 2000. P. 307-10. In such an analysis, inhibitors that are competitive with respect to substrate show a linear increase in IC₅₀ as substrate concentration is increased. The IC₅₀ of inhibitors that are noncompetitive with respect to substrate are independent of substrate concentration. The IC₅₀ of inhibitors that are noncompetitive with respect to substrate are higher at substrate concentrations that are below Km and decrease asymptotically as the substrate concentration increases. When the ATP concentration was varied from 0.25 to 50 μM (˜Km/20 to 10×Km), the IC₅₀ for Compound 1 was 4.6±1.4 nM consistent with noncompetitive inhibition. Similarly when the ERK1 concentration was varied from 0.25 to 10 μM (Km/9 to 4.4×Km), the IC50 for Compound 1 was 2.4±1.9 nM, also consistent with noncompetitive inhibition. Since Compound 1 is a noncompetitive inhibitor of MEK1 with respect to both substrates, the inhibitor appears to bind to the enzyme at a site distinct from those that bind substrates and the Ki is approximately equal to the Ki^{app}.

TABLE 1
Biological and Pharmacologic Properties of Compound 1

| Property | Compound 1 |
|---|---|
| Biochemical Activity | |
| MEK enzyme IC₅₀ (nM)^{a} | 15 |
| Cellular IC₅₀ (nM)^{b} | |
| Colon 26 (mouse) | 0.34 |
| HCT-116 colon (human) | 0.28 |
| MDCK (dog) | 0.31 |
| Pharmacodynamic Activity | |
| EC₅₀ (ng/mL)^{c} | 16.5 |
| EC₉₀ (ng/mL)^{c} | 86 |
| Ex vivo % pERK inhibition at 24 h^{d} | |
| Colon 26 | 76 |
| HT-29 | 79 |
| MiaPaCa2 | 93 |
| Antitumor Activity^{e} | |
| Total daily dose (mg/kg) producing 70% complete tumor | 25 |
| responses | |
| Tumor growth delay in days at above doses | 15.4 |

nd = not determined.
^{a}A cascade assay with raf-1, MEK1, and ERK1 (DELFIA readout) was used to measure enzyme inhibition of MEK1.
^{b}Western assays were used to measure MEK inhibition in cultured cells exposed to the indicated compound for 1 hour.
^{c}Mice bearing the C26 tumor were treated with various doses of Compound 1. For the various doses, the plasma concentrations and decreases in tumor pERK levels were determined. From these values, the plasma concentrations required to inhibit ERK phosphorylation by 50% and 90% (EC₅₀ and EC₉₀) were calculated.
^{d}Tumor-bearing mice were treated orally with Compound 1 at 25 mg/kg. Twenty-four hours after treatment, tumor levels of pERK were measured by Western blot analysis.
^{e}Mice bearing the C26 tumor were treated orally with Compound 1 for 14 days. A complete tumor response represents a decrease in tumor mass below the level of detection. The tumor growth delay is the difference in days for the control and treated tumors to reach a specified evaluation size.

### Example 3

### Cellular Potency and Species Specificity

Compound 1 inhibits phosphorylation of MAP kinase in cultured mouse C26 colon carcinoma cells with an IC₅₀ of 0.33 nM. Species specificity was examined by comparing the potency of Compound 1 in cells of murine, human, and dog origin. One-hour treatment of colon 26 (murine), HCT-116 colon (human), and MDCK (dog) cells with Compound 1 resulted in highly comparable inhibition profiles, as evidenced by IC₅₀ values of 0.34, 0.28, and 0.31 nM, respectively, with respect to inhibition of ERK phosphorylation. This result is consistent with the high degree of sequence homology across species that has been reported for MEK. Cellular activity was 2-fold lower for the S-enantiomer of Compound 1 (IC50=0.83 nM).

### Example 4

### In Vivo Taraet SupDression and Anti-Tumor Efficacv: Oral Dosing

Throughout the preclinical evaluation phase for this agent, the phosphorylation of ERK by activated MEK provided a useful pharmacodynamic marker for assessment of MEK inhibition. For these studies, the C26 tumor model was used. Tumor bearing mice were treated once by oral gavage at doses of 6.25, 12.5, and 25 mg/kg. Twenty-four hours after these single dose treatments, the respective degrees of pERK inhibition were 0%, 59%, and 76%, respectively. The ability of Compound 1 to potently inhibit phosphorylation of ERK was also observed for 2 human tumor xenograft models (HT-29 colon, and MiaPaCa2 Pancreatic). Oral administration of 25 mg/kg Compound 1 resulted in 79% and 93% inhibition of pERK expression 24 hours after dosing, as measured in excised HT-29 and MiaPaCa2 tumors, respectively (Table 2). However, in a third tumor model, BxPC-3, oral administration of 25 mg/kg Compound 1 resulted in only 23% inhibition of pERK expression 24 hours after dosing. The heterogeneity of pharmacodynamic response in different tumor types supports the use of a BID dosing strategy for clinical evaluation of Compound 1.

The PK-PD relationship of Compound 1 to inhibit pERK in C26 tumors was also evaluated following single oral doses of this compound. The doses of Compound 1 ranged from 0.78 to 50 mg/kg. Plasma and tumor samples were collected at 1, 6, 10, and 24 hours after treatment. Plasma Compound 1 concentrations were measured using LC/MS, and tumor pERK was determined by Western blot analysis. The EC₅₀ and EC₉₀ values are the plasma concentrations that are needed to inhibit MEK (as measured by pERK reduction) by 50% and 90%, respectively. The ex vivo potency of Compound 1 in this study was determined to be 16.5 ng/mL (EC₅₀) and 86 ng/mL (EC₉₀) (Table 2).

The maximum tolerated dose (MTD) in BALB/c and SCID mice was 25 mg/kg/d when administered orally, once a day, for 14 days. At the MTD on this regimen, Compound 1 Proved to be highly efficacious against the C26 murine model, as evidenced by a growth delay of 15.4 days and 100% of the animals showing either a complete or partial response (70% and 30%, respectively). As summarized in Table 2, Compound 1 exhibited broad spectrum in vivo antitumor activity against a panel of human tumor xenografts representing colon, pancreatic, and lung origin. Except for the RXF-393 renal cell carcinoma, significant antitumor activity was seen in all tumor models.

TABLE 2
Antitumor Activity of Compound 1 in Preclinical Tumor Models^{a}

| | Tumor | | | | |
|---|---|---|---|---|---|
| Tumor Model | Type | CR^{b} | PR^{c} | T − C (days)^{de} | Tumor Free |
| Colo-205 | Colon | 4/10 | 5/10 | 10.8 | 3/10 |
| HT-29 | Colon | 0/10 | 0/10 | 16.5 | 0/10 |
| MiaPaCa-2 | Pancreatic | 1/9 | 0/9 | 16.8 | 0/9 |
| BxPc-3 | Pancreatic | 2/10 | 8/10 | 20.6 | 0/10 |
| H23 | NSCL | 0/8 | 1/8 | 13.9 | 0/8 |
| RXF-393^{d} | Renal | 0/10 | 0/10 | nd | 0/10 |
| C26^{e} | Colon | 7/10 | 3/10 | 15.4 | 0/10 |
| ZR-75-1^{eg} | Unknown^{f} | 7/8 | 1/8 | 24.2 | 0/8 |

nd = not determined.
^{a}Animals were given an oral dose of 25 mg/kg once a day for 14 days.
^{b}CR = Complete responder.
^{c}PR = Partial responder.
^{d}T − C = difference in days for the treated and control tumors to reach a specified examination size.
^{e}The lowest plasma Compound 1 concentrations associated with meaningful in vivo antitumor efficacy in C26 and ZR-75-1 tumor models in mice were Cₘₐₓ of 260 to 428 ng/mL and AUC₍₀₋₂₄₎ of 1180 to 1880 ng · h/mL.
^{f}Animals were euthanized on day 8 of dosing due to tumor cachexia.
^{g}Human tumor of unknown phenotype. This tumor was originally presumed to be the breast derived ZR-75-1 xenograft. Subsequent analysis revealed that the line was tamoxifen insensitive and that the RNA profile and doubling time was not consistent with ZR-75-1 controls.

An efficacy comparison of Compound 1 administered orally either once a day or BID to C26 tumor-bearing animals was also carried out. As summarized in Table 3, dividing the dose increased toxicity at the top dose. However, at lower, well-tolerated dose levels, there was increased activity as measured by response rate. For example, a total dose of 3.125 mg/kg administered once a day resulted in 1 of 5 complete responders, compared with 4 of 5 complete responders when this dose was split and administered as 1.56 mg/kg twice a day.

TABLE 3
Evaluation of Compound 1 Administered QD Versus BID,
Against Advanced Stage C26 Mouse Colon Carcinoma

| | | Non-specific | % Weight | | | | Net Log₁₀ | Tumor |
|---|---|---|---|---|---|---|---|---|
| Schedule | Dose^{a} | Deaths | Change^{b} | CR^{c} | PR^{d} | T − C^{e} | Kill^{f} | Free^{g} |
| BID, D9-22 | Vehicle | 0/5 | −4.3 (14) | 0/5 | 0/5 | — | — | 0/5 |
| QD, D9-22 | 25 | 0/5 | + | 5/5 | 0/5 | 15.3 | 0.3 | 0/5 |
| QD, D9-22 | 12.5 | 0/5 | + | 5/5 | 0/5 | 15.0 | 0.2 | 0/5 |
| QD, D9-22 | 6.25 | 0/5 | + | 3/5 | 2/5 | 12.9 | 0.0 | 0/5 |
| QD, D9-22 | 3.125 | 0/5 | −4.3 (30) | 1/5 | 4/5 | 12.1 | −0.1 | 0/5 |
| BID, D9-22 | 12.5 | 4/5^{h} | −7.5 (19) | 5/5 Toxic | 0/5 Toxic | Toxic | Toxic | Toxic |
| BID, D9-22 | 6.25 | 0/5 | −4.5 (14) | 5/5 | 0/5 | 15.7 | 0.3 | 0/5 |
| BID, D9-22 | 3.125 | 0/5 | + | 4/5 | 1/5 | 15.2 | 0.3 | 0/5 |
| BID, D9-22 | 1.56 | 0/5 | + | 4/5 | 1/5 | 13.1 | 0.0 | 0/5 |

^{a}Dose is in mg/kg/dose. The vehicle for Compound 1 was 0.5% hydroxypropylmethylcellulose and 0.2% Tween 80 in water. Compound 1 was given orally on schedules listed in the table.
^{b}Maximum treatment related weight loss, expressed as a percent of mean group weight at initial treatment. Value in parentheses indicates the day the maximum weight loss was recorded. A net weight gain is represented by a “+”.
^{c}Complete response is defined as a 100% reduction of initial tumor mass.
^{d}Partial response is defined as at least a 50% reduction of initial tumor mass.
^{e}T − C is the difference, in days, for the median treated and control tumors to reach a fixed evaluation size of 750 mg.
^{f}Net log₁₀ tumor cell kill was calculated from the T − C value.
^{g}Tumor free represents the mice that had an undetectable tumor when the study ended on day 43.
^{h}Mice died on days 18, 20 and 21. All 4 mice had complete tumor regressions at time of death.

A study was also carried out to explore the potential of an intermittent dosing strategy. As summarized in Table 4, animals bearing Colo-205 tumors exhibited the greatest tumor growth delay when dosed daily. Less activity was observed when animals were dosed on intermittent schedules.

TABLE 4
Evaluation of Compound 1 Given on Different Dosing
Schedules Against Human Colon Xenograft, Colo-205

| | | Nonspecific | % Weight | | | | Net Log₁₀ | |
|---|---|---|---|---|---|---|---|---|
| Schedule | Dose^{a} | Deaths | Change^{b} | CR^{c} | PR^{d} | T − C^{e} | kill^{f} | Tumor Free^{g} |
| D19-46 | 0 | 0/6 | −10 (33) | 0/6 | 0/6 | — | — | 0/6 |
| D19-46 | 25 | 0/6 | −2.5 (25) | 5/6 | 1/6 | 63.9 | 2.0 | 1/6 |
| ″ | 12.5 | 0/6 | −1 (22) | 6/6 | 0/6 | 70.1 | 2.3 | 0/6 |
| ″ | 6.3 | 0/6 | + | 2/6 | 4/6 | 46.7 | 1.1 | 0/6 |
| ″ | 3.1 | 0/6 | + | 0/6 | 5/6 | 45.5 | 1.0 | 0/6 |
| D19-23, 26-30 | 25 | 0/6 | −1 (22) | 6/6 | 0/6 | 59.3 | 1.8 | 2/6 |
| 33-37, 40-44 | 12.5 | 0/6 | + | 1/6 | 4/6 | 45.9 | 1.1 | 0/6 |
| ″ | 6.3 | 0/6 | + | 1/6 | 4/6 | 45.1 | 1.1 | 0/6 |
| ″ | 3.1 | 0/6 | −2.5 (22) | 0/6 | 2/6 | 37.8 | 0.7 | 0/6 |
| D19, 22, 25, 28, 31, 34 | 150 | 1/6^{h} | + | 2/5 | 3/5 | 39.9 | 0.7 | 0/5 |
| 37, 40, 43, 46 | 75 | 0/6 | + | 1/6 | 2/6 | 41.5 | 0.8 | 0/6 |
| ″ | 37.5 | 0/6 | + | 0/6 | 0/6 | 37.7 | 0.6 | 0/6 |
| ″ | 18.8 | 0/6 | + | 0/6 | 0/6 | 29.5 | 0.1 | 0/6 |
| D19, 26, 33, 40, 47 | 300 | 3/6ⁱ Toxic | −10 (22) | 2/3 | 1/3 Toxic | 60.3 Toxic | Toxic | 1/3 Toxic |
| | | | | Toxic | | | | |
| ″ | 150 | 0/6 | + | 1/6 | 1/6 | 35.4 | 0.4 | 0/6 |
| ″ | 75 | 0/6 | + | 0/6 | 0/6 | 17.0 | −0.6 | 0/6 |
| ″ | 37.5 | 0/6 | + | 0/6 | 0/6 | 13.1 | −0.8 | 0/6 |

Mean tumor doubling time was 5.6 days.
^{a}Dose is in mg/kg/injection. The vehicle for Compound 1 was 0.5% hydroxypropylmethylcellulose and 0.2% Tween 80 in water. The median mass of the control tumors was 222 mg when dosing was initiated on day 19.
^{b}Maximum treatment related weight loss, expressed as a percent of mean group weight at initial treatment. Value in parentheses indicates the day the maximum weight loss was recorded. A net weight gain is represented by a “+″.
^{c}Complete response is defined as a 100% reduction of initial tumor mass.
^{d}Partial response is defined as at least a 50% reduction of initial tumor mass.
^{e}T − C is the difference in days for the treated and control tumors to reach 750 mg. All tumor free survivors are excluded from T − C calculations.
^{f}Net log₁₀ tumor cell kill was calculated from the T − C value.
^{g}Tumor free represents the mice that had an undetectable tumor as of Day 154.
^{h}Death occurred on Day 21.
ⁱDeaths occurred on Days 23, 28, and 32.

### Example 5

### In Vivo Taraet SupDression and Anti-Tumor Efficacy: Intravenous Dosing

Compound 1 is also highly effective at inhibiting ERK phosphorylation when administered by the IV route. Twenty-four hours after IV administration of 10, 1, or 0.1 mg/kg, significant target suppression was observed in response to all doses (60%, 53%, and 42% inhibition, respectively). Mice appeared to tolerate somewhat higher doses of Compound 1 delivered IV compared with the oral route. The MTD of Compound 1 on an oral daily dosing regimen was determined to be 25 mg/kg. This MTD was established based on data showing that oral daily dosing of 50 mg/kg resulted in 5 out of 8 deaths occurring on the sixth day of treatment, whereas 25 mg/kg delivered daily for 14 days resulted in no deaths or weight loss. In contrast, the LD₁₀ of Compound 1 on an IV regimen was determined to be 100 mg/kg. Efficacy at the respective MTDs was comparable when comparing IV to PO routes of administration, as reflected by T-C values of 15.8 and 12.6 days, respectively, in C26 tumor-bearing mice (Table 5).

TABLE 5
Efficacy of Compound 1 at Its Maximum Tolerated Dose:
Oral versus IV Administration (Colon26 Model)

| Dose | | Weight | | | T − C | Net Log |
|---|---|---|---|---|---|---|
| (mg/kg) | Deaths | Change | CR | PR | (days) | Kill |
| 25 PO^{a} | 0/10 | None | 10/10 | 0/10 | 12.6 | 0.2 |
| 100 IV^{b} | 1/10 | −7.8% | 9/10 | 1/10 | 15.8 | 0.6 |

^{a}Rx QD on Days 11-24
^{b}Rx QD on Days 8-12, 15-19

### Example 6

### Biological Activity of the Carboxylic Acid Metabolite of Compound 1

The compound of formula 2 (Compound @2, the carboxylic acid and major metabolite of Compound 1, was found to be comparably efficacious with the parent compound when assayed against purified MEK1, as evidenced by IC₅₀ values of 66 nM and 15 nM, respectively. 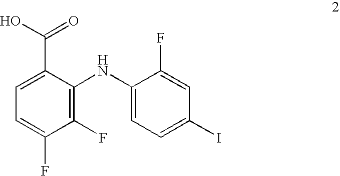
However, Compound 2 was found to be significantly less effective at reducing ERK phosphorylation in tumor cells grown in culture in the presence of 10% serum, exhibiting an IC₅₀ of 1.3 μM compared with 0.59 nM for the parent compound evaluated in the same test. The metabolite was also tested head to head in a pharmacodynamic assay using C26 tumor-bearing animals. Compound 2 was found to possess biological activity as evidenced by nearly complete (98%) target suppression of pERK in tumors 2 hours after dosing with 75 mg/kg IV. However, by 24 hours after dosing, activity had returned to control levels. In comparison, the parent compound Compound 1 still resulted in 60% inhibition at the 24-hour time point at doses as low as 25 mg/kg (also administered IV). While biologically active, concentrations of metabolite associated with target suppression far exceed concentrations of metabolite achieved following administration of parent.

### Example 7

### Pharmacokinetics-Pharmacodynamics of Compound 1

The PK-PD relationship of Compound 1 to inhibit phosphorylation of MAPK was evaluated in preclinical tumor models in mice with C26 colon tumor (a murine-derived tumor) and in tumor xenograft models with human-derived tumors (BxPc3, Mia-Pa-Ca 2, Colo-205, and HT-29). The IC₅₀ value of Compound 1 to inhibit phosphorylation of MAPK was 16.5 ng/mL in C26 colon tumor model. The ICm values of Compound 1 to inhibit the human-derived tumor xenograft models were 40.9 (BxPc3), 5.17 (Mia-Pa-Ca 2), 11.5 (Colo-205) and 53.5 (HT-29) ng/mL. Based on the data from BxPc3, Colo-205, and HT-29 tumor xenograft models, it is projected that maintaining a Css, avg concentration of 5 times the IC₅₀ value will result in near maximal suppression of phosphorylation of MAPK throughout the dosing interval. Therefore, the projected therapeutic concentrations (steady-state average plasma concentrations, Css, avg) in humans range from approximately 60 ng/mL to 270 ng/mL (or approximately 0.125 to 0.56 μM), which corresponds to AUC(₀₂₄) values of 1440 to 6480 ng·h/mL.

### Example 8

### Characterization of in vitro Metabolites of [¹⁴C] Labeled Compound 1 in Rat, Dog, Monkey, and Human Crvopreserved Hepatocvtes and Microsomes

As part of the preclinical evaluation process, metabolites of [¹⁴C] labeled Compound 1 have been determined in vitro, in four different species: rat, dog, monkey, and humans.

Cross species incubations were performed in cryopreserved hepatocytes and liver microsomes at a substrate concentration of 10 μM of Compound 1 labeled with [¹⁴C] at the carbonyl carbon adjacent to the 3,4-difluoro ring. Incubations were performed in hepatocytes for 3 hours and liver microsomes for 1.5 hours and were stopped with the addition of methanol. HPLC, in conjuction with ionspray 3D iontrap mass spectrometry, and simultaneous radioactive monitoring were used to detect and identify the resulting metabolites.

Metabolite identification of radiolabeled Compound 1 was performed after incubation with rat, dog, monkey and human cryopreserved hepatocytes and liver microsomes. As shown in Table 1 and depicted in Scheme 1, major metabolites formed in hepatocytes include multiple glucuronides of Compound 1: M 6 and M 7, 27 and 18% in humans, 21 and 23% in monkeys, respectively; M 10, 20% in monkeys and 16% in dogs, respectively. Besides glucuronidation, metabolites derived from other clearance pathways are also observed. For instance, M 15 (acid cleavage product) accounts for 11% of the total radioactivity in the human chromatogram.

Scheme 1.

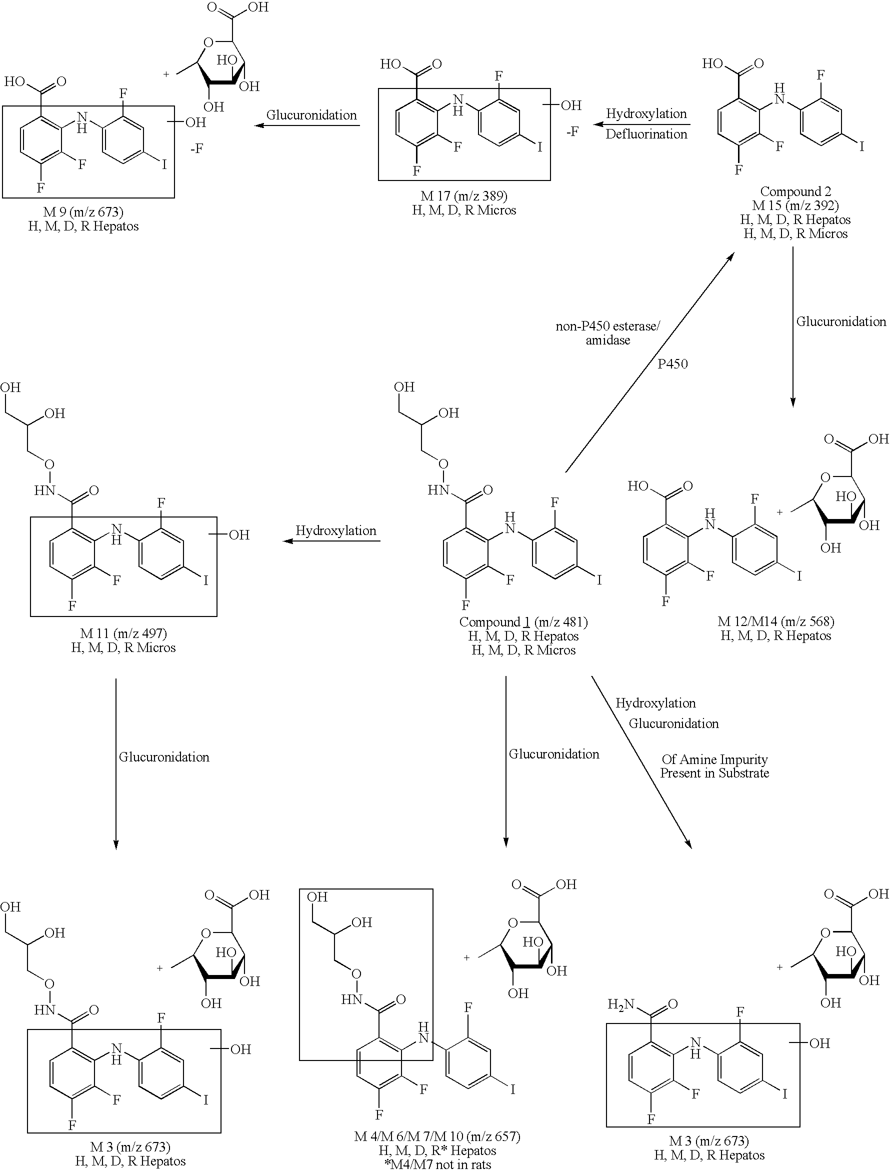

Major metabolites formed in microsomes include an acid cleavage product at the amide bond (M 15, 9% in human) that forms in the absence of NADPH and after pre-incubation with the mechanism-based pan P450 inhibitor aminobenzotriazole, suggesting non-P450 metabolism. This acid cleavage product is further metabolized to form hydroxylated (M 11, 16% in rat) and hydroxylated, defluorinated products (M 17, 19% in rat).

Taken together, results from in vitro incubations indicate multiple clearance pathways for Compound 1.

TABLE 6
Liquid Chromatography-Mass Spectrometry and
Metabolite Detection Conditions

| Equipment | Model and Vendor |
|---|---|
| HPLC Pump | Agilent 1100 (Hewlett Packard) |
| Autosampler | Agilent 1100 (Hewlett Packard) |
| MW UV Detector | Agilent 1100 (Hewlett Packard) |
| UV Wavelength | 280 nm |
| Column | Supelco, Discovery HS C18, 150 × 4.6 mm, 5μ |
| Guard Column | Supelguard, Discovery 18, 20 × 4 mm, 5 μ |
| Mass Spectrometer | Quadrupole Ion Trap LCQ-Advantage |
| | (ThermoQuest, San Jose, CA) |
| Radioactivity Detection | IN/US β-RAM, Model 2 equipped with a |
| | 500 □L flow cell (Laura 3 Radio HPLC |
| | software version 3.0, Lablogic Systems Ltd) |

| Mass Spectrometer Parameter | Setting |
|---|---|
| Ionization Mode | Negative |
| Source Voltage | 3.0 kV |
| Capillary Voltage | 4.0 V |
| Tube Lens Offset | 15.0 V |
| Capillary Temperature | 250.0° C. |
| Sheath Gas Flow | 80 (arb.) |
| Aux Gas Flow | 20 (arb.) |
| Activation Amplitude | 27% |
| Activation Q | 0.25 |
| Activation Time | 30 ms |

TABLE 7
Gradient Conditions for Liquid Chromatography

| Total | Solvent A | Solvent B | Flow Rate | |
|---|---|---|---|---|
| Time (min) | (%) | (%) | (mL/min | Event |
| 0 | 75 | 25 | 1 | Initial |
| | | | | (Divert to Waste |
| | | | | 0.0-3.0 Min.) |
| 28 | 75 | 25 | 1 | Hold |
| 33 | 50 | 50 | 1 | Linear Gradient |
| 38 | 50 | 50 | 1 | Hold |
| 43 | 10 | 90 | 1 | Linear Gradient |
| 48 | 10 | 90 | 1 | Hold |
| 53 | 75 | 25 | 1 | Linear Gradient |
| 58 | 75 | 25 | 1 | Hold |
| | | | | (Divert to Waste |
| | | | | 55.5-58.0 Min.) |

TABLE 8
Metabolites of [C-14] Compound 1 detected after incubation
(10 μM, 180 mins) with cryopreserved hepatocytes

| | | β-Ram | | | | | |
|---|---|---|---|---|---|---|---|
| | | Retention | | | | | |
| Metabolite/ | Mass/Charge | Time | % ROI | | | | Proposed |
| Label | Ratio [M − H]⁻ | (Min) | Human | Monkey | Dog | Rat | Identity |
| M 3 | 673 | 10.28 | 3.69 | 1.19 | 0.85 | 2.49 | Compound 1 Hydroxylation + Glucuronidation |
| M 4 | 657 | 15.08 | 3.73 | 0.86* | 4.61 | — | Compound 1 Glucuronidation |
| M 5 | 583 | 17.39 | 2.31 | 1.90 | 3.67 | 1.16 | Amide Impurity Hydroxylation + Glucuronidation |
| M 6 | 657 | 19.14 | 27.08 | 21.34 | 5.95 | 6.45 | Compound 1 Glucuronidation |
| M 7 | 657 | 20.36 | 18.42 | 22.49 | 4.75 | — | Compound 1 Glucuronidation |
| M 8 | N.D. | 28.48 | 1.45 | 0.66\hat{ } | 3.31 | — | N.D. |
| M 9 | 565 | 30.05 | 2.21 | 1.74 | 4.28 | 5.64 | Compound 2 Hydroxylation, |
| | | | | | | | Defluorination, +Glucuronidation |
| M 10 | 657 | 32.27 | 2.46 | 19.93 | 15.48 | 0.82* | Compound 1 Glucuronidation |
| M 12 | 568 | 33.46 | 6.32 | 1.65 | 1.34 | 0.88* | Compound 2 Glucuronidation |
| M 14 | 568 | 34.19 | 4.35 | 0.84 | 0.86 | 0.83\hat{ } | Compound 2 Glucuronidation |
| M 15 | 392 | 34.53 | 11.07 | 1.72 | 2.46 | 4.31 | Compound 2 (acid cleavage |
| Compound 2 | | | | | | | product) |
| Compound 1 | 481 | 35.31 | 15.68 | 23.46 | 50.45 | 72.06 | Compound 1 |
| M 16 | N.D. | 37.45 | — | 1.38 | 1.99 | 5.57 | N.D. |

N.D. = Not Determined
*= Determined from n = 2 ROI's from independent profiles/injections.
\hat{ }= Determined from n = 1 ROI's from independent profiles/injections.
Note:
All values determined from n = 3 ROI's from independent profiles/injections unless indicated otherwise.
Initial stock of ¹⁴C PD 0325901 contained 2.49% of amide impurity which was further metabolized to M 5.

TABLE 9
Metabolites of [C-14] Compound 1 detected after
incubation (10 μM, 90 mins) with liver microsomes

| | | β-Ram | | | | | |
|---|---|---|---|---|---|---|---|
| | | Retention | | | | | |
| Metabolite/ | Mass/Charge | Time | % ROI | | | | Proposed |
| Label | Ratio [M − H]⁻ | (Min) | Human | Monkey | Dog | Rat | Identity |
| M 1 | N.D. | 3.01 | 1.28* | 1.38 | 1.24 | 1.59 | N.D. |
| M 2 | N.D. | 3.47 | 1.15\hat{ } | 0.91 | 0.82* | 2.78 | N.D. |
| M 11 | 497 | 32.34 | 4.08 | 4.54 | 2.36 | 15.72 | Compound 1 Hydroxylation |
| M 13 | N.D. | 34.13 | 2.00 | 6.37 | 2.66 | 3.19 | N.D. |
| M 15 | 392 | 35.59 | 8.73 | 5.24 | 4.12 | 1.86 | Compound 2 (acid cleavage |
| Compound 2 | | | | | | | product) |
| Compound 1 | 481 | 36.44 | 77.36 | 71.38 | 81.16 | 52.30 | Compound 1 |
| M 17 | 389 | 38.24 | 5.40 | 6.47 | 5.09 | 18.63 | Compound 2 Hydroxylation + Defluorination |
| M 18 | N.D. | 41.38 | 1.90 | 3.71 | 8.45 | 2.99 | N.D. |

N.D. = Not Determined
*= Determined from n = 2 ROI's from independent profiles/injections.
\hat{ }= Determined from n = 1 ROI's from independent profiles/injections.
Note:
All values determined from n = 3 ROI's from independent profiles/injections unless indicated otherwise.

### Example 9

### Phase 1 Clinical Study of Compound 1 in Patients with Advanced Cancer

During an ongoing Phase 1/2 study 42 Patients have been treated with Compound 1. Oral dose escalation was conducted from 1 mg QD through 30 mg BID days 1-21 of 28-day cycles in patients with breast, colon, nonsmall cell lung cancer (NSCLC) or melanoma. Continuous dosing in 28-day cycles was subsequently tested.

Tumor tissue was assessed by immunohistochemistry (IHC) for pERK at baseline (BL) and Day 15 of Cycle 1 as described in Example 10 and for the amount of Ki67 as described in Example 11. Pharmacokinetic (PK) samples were obtained on Days 1 and 21 in all patients and also on Day 1 of Cycle 2 in patients participating in a 2-way crossover food effect component. Due to elevated serum phosphorus with corresponding soft tissue mineralization observed in rats, serum Ca, P and (Ca×P) product were monitored closely.

Drug related toxicities seen included acneiform rash, mucositis, blurry vision, confusion, edema, diarrhea, and fatigue. Grade 1-2 acneiform skin rash occurred in a significant portion of patients, and Grade 3 rashes were seen at doses above 15 mg BID. This has been managed with minocycline 50-100 mg per day. Two episodes of syncope occurred, possibly drug related, and considered DLT at 30 mg BID. Reversible grade 1-2 visual effects including blurred vision and halos have been seen. These typically were less severe later in a drug cycle, during intercycle drug holidays, and during subsequent cycles. Grade 1-2 dependent and facial edema was seen intermittently with all 6ose levels. Grade 1-2 diarrhea was seen and responded well to Imodium.

The maximum administered dose (MAD) was 30 mg BID, and the MTD when dosing 21 days of a 28-day cycle was determined to be 20 mg BID, secondary to 1/6 cases of DLT Grade 3 acneiform skin rash and 2/6 cases of DLT syncope (Grade 3 by CTC AE 3.0) at 30 mg BID on 21 day dosing in 28-day cycles. Cohorts of 6 Patients each were subsequently treated at 20 mg bid and 15 mg bid with dosing continuously over 28-day cycles. A DLT was seen in one patient in each cohort (grade 3 acneiform skin rash).

Of 42 Patients treated, 2 Partial responses (melanoma) and 10 Patients with stable disease have been reported. There was no notable effect on serum (C×P) product.

### Example 10

### Immunohistochemical Analysis of Phosohorylated ERK Protein (PERK) in Paired Serial Biopsy Specimens from Patients Treated with Compound 1

The ERK protein (also known as MAP kinase, or MAPK) is a substrate for MEK activity, and thus reduction in the phosphorylation of the ERK protein is indicative of a reduction in MEK activity. The phosphorylation status of ERK can be assessed through antibody-based detection methods, utilizing phosphorylation site-specific antibodies. In a clinical study of cancer patients treated with Compound 1, biopsy specimens were collected immediately before the onset of treatment and on the 15th day of daily dosing with Compound 1. The biopsies were collected from tumor lesions amenable to either excisional or core needle biopsy procedures; the tissue specimens were immediately placed into 10% neutral-buffered formalin solution for fixation (nominally 6-8 hours, but no more than 24 hours in formalin). The fixed tissues were then transferred to 70% ethanol solution and submitted to the histopathology assay lab for immunohistochemistry (IHC) analysis of both pERK and the proliferation marker Ki67 (Example 11). The biopsy tissues were paraffin-embedded following standard tissue processing. The antibody used for detection of pERK was the mouse monoclonal anti-MAP kinase (activated) antibody, manufactured by Sigma-Aldrich Co. (catalog number: M8159). This antibody is reacts specifically with the dually phosphorylated form of MAPK (both the ERK-1 and ERK-2 forms). Tissue sections prepared from the paraffin blocks were pretreated with Heat-induced Epitope Retrieval conditions (3 minutes at 120° C.) prior to the detection procedure. A DAKO Envision plus kit was used for visualization of the antibody bound to sections, with DAB as the chromogen. A Biogenex Autostainer system was used in the staining procedure (with hematoxylin counterstaining). The extent of staining in individual sections was assessed by pathologist review, with scoring based on the relative staining color intensities in tumor cells in each section (prior to biomarker evaluation, a hematoxylin-eosin stained slide from each biopsy tissue was used for evaluation of whether and to what extent tumor cells were present in a given biopsy specimen; tissues in which little or no tumor cell content was observed were not included in the analysis). Staining intensity was grouped in categories from 0 to 3+, with 0 being little or no staining, and 3 being the most intense staining. The final score for each section was given in the form of an H-score, which was calculated as follows: 3×(% of cells with 3+staining)+2×(% of cells with 2+staining)+1×(% of cells with 1+staining). For each biopsy specimen, 2 sections were analyzed and the average of these was taken for the pERK H-score. In addition to H-scores, the total % of positive cells was also recorded. Comparison of pre-treatment biopsies to the post-treatment biopsy from the same patient was calculated based on H-scores using the following formula: the negative of ((pre-post)/pre)×100; to give the percentage difference in P-ERK associated with treatment with Compound 1.

### Example 11

### Immunohistochemical Analysis of Cell Proliferation Marker Ki67 in Paired Serial Biopsy Specimens from Patients Treated with Compound 1

The same biopsy specimens, as analyzed for pERK. staining described in Example 10, were also assessed for the amount of Ki67 detectable by immunohistochemistry. The Ki67 marker is considered an indicator of cell proliferation and thus reduction in Ki67 staining levels may be correlated with anti-tumor activity of therapeutic agents. Tissue sections from the paraffin-embedded biopsy tissues were pretreated with Heat-induced Epitope Retrieval (in this case, microwaving for 3 minutes) prior to the Ki67 detection procedure. The antibody used was the mouse monoclonal Ki67/MM1 manufactured by Novocastra Laboratories, Ltd. (catalog number: NCL-Ki67-MM1). A Biogenex Supersensitive DAB/HRP detection kit was used for visualization of the antibody bound to sections, with DAB as the chromogen. A Biogenex Autostainer system was used in the staining procedure (with hematoxylin counterstaining). The extent of staining in individual sections was assessed by pathologist review, with scoring based on the relative staining color intensities in tumor cells in each section. Scoring of staining intensity was performed in same manner as for pERK assay, with H-scores generated for each section. For Ki67, only 1 section was assayed for each biopsy specimen. The percentage difference in Ki67 scores associated with Compound 1 was calculated in the same manner as for pERK.

### Example 12

### Pharmacokinetics (PK), Pharmacodynamics (PD) and Product Metabolism of Compound 1 Following Multiple Oral Doses to Advanced Cancer Patients

The first-in-human Phase 1/2 trial employed an open-label, dose-escalating design where patients with various advanced solid tumors were treated orally with Compound 1 (QD or BID) for 21 or 28 days in 28-day cycles. Pharmacokinetic data are presented for the first 38 subjects. Dose escalation was conducted from 1 mg QD, and 1 mg BID through 30 mg BID. Continuous dosing at 20 mg BID for 56 days was subsequently tested.

Serial blood samples were collected on Days 1 and 21 of Cycle 1, and Day 1 of Cycle 2 for pharmacokinetic evaluation of the parent Compound 1, its major metabolite Compound 2 and its S-enantiomer, N-[(S)-2,3-dihydroxy-propoxy]-3,4-difluoro-2-(2-fluoro-4-iodo-phenylamino)-benzamide (Compound 1. The effect of food on the PK of Compound 1 was evaluated in 16 Patients (Day 1 of Cycles 1 & 2).

A liquid chromatographic method with tandem mass spectrometry detection (LC/MS/MS) was used to determine the concentrations of Compound 1, Compound 3, and Compound 2 in human plasma with EDTA as an anticoagulant. Blood samples were collected from patients into a purple-top potassium EDTA Vacutainer tube. Following mixing with gentle inversion (8-10 times), tubes were centrifuged within 20 minutes at 1000×g for 15 minutes, to harvest plasma. Plasma samples were stored at −20° C. until analysis.

Compound 1 and its isomer Compound 3 along with the metabolite Compound 2 were extracted from EDTA human plasma using methyl-t-butyl ether/ethyl alcohol (95:5 v/v), followed by quantitation using LC/MS/MS with Turbo ion spray in the negative ion mode. The range of quantitation for Compound 1, Compound 3, and Compound 2 was 0.100 to 100 ng/mL. This assay required a 0.200 mL aliquot of plasma. The method procedures are as following:

Sample Preparation

To a 0.2 mL aliquot of plasma samples, added 0.050 mL of working solution of internal standard (IS) (18.75 ng/mL [2 H₃]-Compound 1, 50.0 ng/mL [2 H₃]-Compound 2 in 60:40 methanol/water, v/v). Added 0.075 mL 0.2% formic acid in water, and gently vortexed for 30 seconds. Added 0.650 mL 95:5 methyl-t-butyl ether/ethyl alcohol, v/v. Vortexed using the multitube vortexer for 1 minute. Repeated this step 3 more times. After vortexing step, centrifuged at 4° C. for 20 minutes at 4000 rpm. After centrifugation, transferred approximately 0.550 mL to a 1-mL 96-well polypropylene plate. Evaporated to dryness under nitrogen at 30° C. to 35° C. Reconstituted sample with 0.075 mL 70:30 methanolwater containing 0.1% acetic acid. Vortexed using the multitube vortexer for 1 minute. Centrifuged for 5 minutes at 4000 rpm. Injected 1 to 10 PL of sample extract.

Chromatographic Conditions

| | |
|---|---|
| Analytical Column: | CHIRALPAK AD-RH, 0.46 × 15 cm, 5 μ |
| Guard Column: | MetaGuard 2.0 mm Polaris C18-A, 5 μ |
| Mobile Phase: | Mobile Phase A: 0.1% acetic acid in water, v/v |
| | Mobile Phase B: 90:10 acetonitrile/isopropanol, |
| | v/v |
| | 17:83 Mobile Phase A/Mobile Phase B |
| Needle Flush Solvent: | 300:300:400:1 Acetonitrile/ |
| | isopropanol/water/acetic acid, v/v/v/v |
| Injector Loop: | 50 μL |
| Pump Program: | Isocratic |
| Flow Rate: | 0.750 mL/min |
| Column Temperature: | 35° C. |
| Injection Volume: | 1-10 μL |
| Approximate | Compound 1 2.6 to 3.2 minutes |
| Retention Times: | Compound 3 3.2 to 3.8 minutes |
| | Compound 2 2.7 to 3.3 minutes |

Mass Spectrometry

| | |
|---|---|
| Mass Spectrometer: | Sciex API 4000 |
| Ionization Mode: | Turbo ion spray/negative ion mode |
| Acquisition Mode: | MRM |
| Pause Time: | 5 ms |

| | | | Dwell^{a} | CE^{a} | DP^{a} | CXP^{a} |
|---|---|---|---|---|---|---|
| Compound | Q1 m/z | Q3 m/z | (ms) | (V) | (V) | (V) |
| 1 | 481.0 (±0.5) | 389.0 (±0.5) | 125 | −25 | −55 | −15 |
| 3 | 481.0 (±0.5) | 389.0 (±0.5) | 125 | −25 | −55 | −15 |
| 2 | 392.0 (±0.5) | 328.0 (±0.5) | 125 | −21 | −42 | −15 |

^{a}Instrument dependent, may be adjusted to optimize performance.

Run Time: 6.0 or 12.0 minutes, depending on column condition
   Quantitation

PE Sciex Analyst software (Version 1.2) was used to measure peak areas. Watson LIMS (Version 6.4.0.04, CPL #63) was used for data reduction. Peak areas of Compound 1 to [²H₃]-Compound 1 (IS) peak areas, Compound 3 Peak areas to [²H₃]-Compound 1 Peak areas, and Compound 2 Peak area to [²H₃]-Compound 2 Peak areas were calculated. Calibration curves were constructed using peak area ratios (PARS) of the calibration samples by applying 1/concentration² weighted quadratic regression analysis. All concentrations were then calculated from their PARS against their respective calibration line.

The following results are based on preliminary pharmacokinetic parameters from the 38 subjects, estimated using nominal collection times and quality-controlled, non quality-assured bioanalytical data. Pharmacokinetic parameters were determined by the non-compartmental method using WINNONLIN Professional Edition (Version 4.1). The liner trapezoidal method was used to calculate area under the concentration-time curve from 0 to 24 hours (AUC₀₋₂₄). The half-life of the terminal phase (t_{1/2}) in the plasma concentration-time curve was calculated from t_{1/2}=0.693/λ₂, where λ₂ was estimated by linear regression using the last 3 concentration data points (r²>0.9). The maximum observed concentration (Cₘₐₓ) was obtained by inspection of the concentration data. The time to reach the Cₘₐₓ was the first time at which Cₘₐₓ is observed and obtained by inspection of the data. The pre-dose concentration (C_{trough}) was obtained by inspection of the concentration data. The average concentration at steady state (C_{ss.avg}) was calculated from AUC₂₋₂₄ on Day 21 divided by the daily dosing interval (24 hours).

Preliminary plasma pharmacokinetic parameters of Compound 1 are presented in Table 10. Compound 1 administered in the fasted state was absorbed rapidly, with peak plasma concentrations occurring within 1 to 2 hours after dosing. Under fasting conditions, both peak plasma levels (Cₘₐₓ) and area under the curve (AUC) were roughly dose proportional in a range of 1 mg QD or BID -30 mg BID. Plasma concentrations declined with an elimination plasma half-life generally ranging between 5 to 14 hours. After 21-day multiple oral BID dosing, AUC of Compound 1 increased slightly with an accumulation ratio of 1.1-2.2. The inter-subject variability in Compound 1 Pharmacokinetics was evaluated in this subject population. The overall coefficients of variation (CV) for AUC(O2₄) in the fasted state was 39%.

TABLE 10
Plasma Pharmacokinetic Parameters of Compound 1
(Mean Estimates With Coefficient of Variation, CV %) on
Day 21 of Cycle 1 (C1D21) under Fasting Conditions

| | Number | Cₘₐₓ | Tₘₐₓ | C_{trough} | AUC₍₀₋₂₄₎ | T_{1/2} | |
|---|---|---|---|---|---|---|---|
| Dose Cohort | of patients | (ng/mL) | (hr) | (ng/mL) | (ng · hr/mL) | (hr) | AR* |
| 1 mg QD | 4 | 22.3 | 2 | 3.00 | 143 | 13.9 | 1.6 (22) |
| | | (42) | (67) | (48) | (19) | (7) | n = 4 |
| 1 mg BID | 2 | 41.1 | 1 | 9.17 | 338 | ** | 2.1 |
| | | | | | | | n = 1 |
| 2 mg BID | 4 | 152 | 1 | 27.4 | 1161 | ** | — |
| | | (30) | (0) | (49) | (50) | | |
| 4 mg BID | 3 | 208 | 1 | 28.0 | 1581 | 7.6 | 1.5 (17) |
| | | (23) | (0) | (6) | (22) | (31) | n = 3 |
| 15 mg BID | 3 | 706 | 1 | 142 | 5687 | 12.6 | 1.1 |
| | | (73) | (0) | (24) | (49) | n = 3 | n = 2 |
| 20 mg BID | 3 | 766 | 2 | 112 | 6987 | 8.3 | 1.3 |
| | | (12) | (35) | (67) | (45) | (24) | n = 1 |
| 30 mg BID | 2 | 1640 | 2 | 157 | 12460 | 4.8 | 1.1 |
| | | | | | | | n = 2 |
| 20 mg BID | 5 | 989 | 2 | 83.7 | 6572 | 5.1 | 1.4 (11) |
| continuous | | (53) | (56) | (26) | (16) | (37) | n = 5 |

*AR (accumulation ratio) = AUC₍₀₋₂₄₎ (C1D21)/AUC₍₀₋₂₄₎ (C1D1 fasted)
** Half-life estimates are not available for some patients due to insufficient collection time points

After oral administration, a major circulating metabolite of Compound 1 was the carboxylic acid metabolite, Compound 2. The AUC of Compound 2 was approximately 66% and 120% (medians) of the parent following single and multiple dosing of Compound 1 in human plasma, respectively. The preliminary plasma pharmacokinetic parameters of Compound 2 are summarized in Table 11. Following oral administration of Compound 1 under fasting conditions, both AUC and Cₘₐₓ of Compound 2 were generally increased with doses ranging from 1 mg QD or BID -30 mg BID. The terminal plasma half-life of Compound 2 was longer than that of the parent drug. After 21-day multiple BID dosing, AUC of Compound 2 increased 2.0-5.0 fold.

TABLE 11
Plasma Pharmacokinetic Parameters of Compound 2, a
major metabolite (Mean Estimates With Coefficient of Variation,
CV %) on Day 21 of Cycle 1 (C1D21) under Fasting Conditions

| | Number | Cₘₐₓ | Tₘₐₓ | C_{trough} | AUC₍₀₋₂₄₎ | T_{1/2} | |
|---|---|---|---|---|---|---|---|
| Dose Cohort | of patients | (ng/mL) | (hr) | (ng/mL) | (ng · hr/mL) | (hr) | AR* |
| 1 mg QD | 4 | 5.98 | 5 | 3.55 | 109 | 32 | 2.0 (7) |
| | | (43) | (52) | (57) | (54) | (46) | n = 4 |
| 1 mg BID | 2 | 15.4 | 1 | 10.5 | 281 | 20 | — |
| 2 mg BID | 4 | 54.3 | 2 | 34.6 | 994 | 27 | — |
| | | (47) | (86) | (56) | (56) | (86) | |
| 4 mg BID | 3 | 166 | 2 | 104 | 2996 | 15 | 5.0 |
| | | (44) | (50) | (50) | (50) | (25) | n = 2 |
| 15 mg BID | 3 | 405 | 3.7 | 321 | 8430 | 71 | 3.5 |
| | | (58) | (57) | (45) | (53) | (55) | n = 2 |
| 20 mg BID | 3 | 306 | 3 | 194 | 5907 | 14 | 2.7 |
| | | (43) | (35) | (57) | (46) | (41) | n = 1 |
| 30 mg BID | 2 | 604 | 2 | 420 | 11740 | 20 | 2.7 |
| | | | | | | | n = 2 |
| 20 mg BID | 5 | 352 | 2 | 182 | 6060 | 30 | 2.6 (21) |
| continuous | | (29) | (38) | (14) | (26) | (120) | n = 5 |

*AR (accumulation ratio) = AUC₍₀₋₂₄₎ (C1D21)/AUC₍₀₋₂₄₎ (C1D1 fasted)
** Half-life estimates are not reliable due to insufficient collection time points

A pilot food effect evaluation was conducted on Day 1 of Cycle 1 (ClDl) and Day 1 of Cycle 2 (C1D2). Sixteen subjects completed the pilot food evaluation and their data are presented in Table 12. When Compound 1 was taken with a high fat meal, peak plasma levels were delayed for about 2-7 hours with a decrease in Cₘₐₓ, suggesting that the absorption rate of Compound 1 was reduced by food ingestion. However, the effect of food on Compound 1 AUC was variable. The coefficients of variation (CV) for AUC₍₀₋₂₄₎ in the fed state was 78%, about twice of that in the fasted state, indicating that food ingestion increased pharmacokinetic variability.

TABLE 12
Pilot Food Effect Assessment: Comparison of Plasma
Pharmacokinetics of Compound 1 in the Fed versus Fasted State

| | | Cₘₐₓ (ng/mL) | | | AUC₍₀₋₂₄₎ (ng · hr/mL) | | | Tₘₐₓ (hr) | |
|---|---|---|---|---|---|---|---|---|---|
| Dose | Patient | Fed | Fasted | Change % | Fed | Fasted | Change % | Fed | Fasted |
| 1 mg | 1 | 33.4 | 181 | −82 | 244 | 998 | −76 | 3 | 1 |
| BID | 2 | 56.4 | 21.6 | 161 | 652 | 133 | 390 | 4 | 1 |
| 2 mg | 3 | 56.7 | 88.7 | −36 | 456 | 454 | 1 | 4 | 1 |
| BID | 4 | 29.5 | 77.5 | −62 | 369 | 594 | −38 | 6 | 1 |
| | 5 | 122 | 173 | −29 | 2080 | 874 | 138 | 8 | 1 |
| | 6 | 52.1 | 71.9 | −28 | 914 | 526 | 74 | 4 | 2 |
| 4 mg | 7 | 49.1 | 65.5 | −25 | 654 | 705 | −7 | 6 | 3 |
| BID | 8 | 80.7 | 157 | −49 | 849 | 1060 | −20 | 4 | 1 |
| | 9 | 58.4 | 114 | −49 | 590 | 721 | −18 | 6 | 1 |
| | 10 | 68.0 | 157 | −57 | 754 | 919 | −18 | 3 | 1 |
| 15 mg | 11 | 459 | 1120 | −59 | 4700 | 5460 | −14 | 4 | 1 |
| BID | 12 | 92.0 | 464 | −80 | 957 | 2880 | −67 | 4 | 1 |
| | 13 | 319 | 641 | −50 | 3580 | 4880 | −27 | 4 | 1 |
| 20 mg | 14 | 358 | 839 | −57 | 4060 | 4340 | −6 | 4 | 1 |
| BID | 15 | 797 | 1180 | −32 | 6700 | 6820 | −2 | 3 | 1 |
| | 16 | 393 | 596 | −34 | 2860 | 3360 | −15 | 2 | 2 |

Compound 1 was dosed as purified R-enantiomer. The in vivo interconversion from R-(Compound 1) to S-enantiomer (Compound 1 was evaluated in twenty-four subjects across 8 different dosing regimens. The average S-to-R ratio for AUC on Day 21 of cycle 1 was low, 0.03 (CV 38%); this excludes two outlier subjects with ratios of 12 and 21% each from the 1 mg BID cohort.

In summary, the plasma pharmacokinetics of Compound 1 is described by rapid absorption, with peak concentrations occurring within 1-2 hours of dosing, generally dose-proportional changes in exposures, and an elimination half-life of 5-16 hours. Food appeared to reduce Compound 1 Peak plasma concentrations, but the effect on AUC was variable. Plasma pharmacokinetics for the major circulation metabolite Compound 1 was characterized by a longer half-life and up to 120% higher plasma exposures than the parent.

PD markers of MEK1/2 activity (pERK) and cell proliferation (Ki67) were assessed by quantitative immunohistochemistry in tumor biopsies obtained at baseline and on Day 15 (2-4 hours after dosing). Strong tumor pERK suppression was observed across different dose regimens ranging from 1 mg QD or BID to 30 mg BID; in results being gathered from the ongoing study, the average pERK decline was 68% over all doses (N=23), and 75% from 2 mg BID to 30 mg BID (N=20), relative to baseline. When only those cases with baseline pERK score above 20 are considered, the average pERK decline was 63% over all dose groups from 1 mg QD to 30 mg BID (N=19), and 74% from 2 mg BID to 30 mg BID (N=14). Ki67 was also affected over this dose range; the average Ki67 decline was 31% over dose ranges from 1 mg QD to 30 mg BID (N=22) and 46% from 2 mg BID to 30 mg BID (N=19). When only those cases with baseline Ki67 scores above 20 are considered, the average Ki67 decline was 39% across all dose groups (N=18), and 49% over the 2 mg BID to the 30 mg BID groups (N=13). As a result of these investigations of pERK in tumor biopsies, it has been shown that Compound 1 led to suppression of pERK levels in tumors at all doses tested, and particularly at doses of 2 mg or greater. The proliferation marker Ki67 also showed a decline in post-treatment tumors and a similar dose effect. Table 13 represents data for the ongoing Phase 1/2 study.

TABLE 13
Data for the Ongoing Phase ½ Study*

| | | | | pERK | Compound 1 | | |
|---|---|---|---|---|---|---|---|
| Patient | | | pERK | % Reduction | AUC₍₀₋₂₄₎, | Compound 1 | |
| No./Type | | pERK | Post- | from | Day 21 | Css, avg | Clinical |
| of Cancer | Dose | Baseline | Treatment | Baseline | (ng * hr/mL) | (ng/mL) | Response |
| 1 | 1 mg QD | 30 | 78 | 0 | 112 | 4.7 | |
| Melanoma | | | | | | | |
| 2 | 1 mg QD | 140 | 135 | −4 | 130 | 5.4 | |
| Breast | | | | | | | |
| 3 | 1 mg QD | 40 | 3 | −93 | 170 | 7.1 | |
| Melanoma | | | | | | | |
| 4 | 1 mg BID | 130 | 45 | −65 | ND | ND | |
| Melanoma | | | | | | | |
| 5 | 1 mg BID | 40 | 40 | 0 | 275 | 11 | |
| Melanoma | | | | | | | |
| 6 | 2 mg BID | 290 | 10 | −97 | 785 | 33 | stable |
| Melanoma | | | | | | | disease |
| 7 | 2 mg BID | 4 | 12 | 0 | 898 | 37 | stable |
| Colon | | | | | | | disease |
| 8 | 2 mg BID | 14 | 0 | −100 | 2020 | 84 | |
| Melanoma | | | | | | | |
| 9 | 2 mg BID | 150 | 8 | −95 | 943 | 39 | |
| Lung | | | | | | | |
| 10 | 4 mg BID | 60 | 0 | −100 | 1974 | 82 | stable |
| Lung | | | | | | | disease |
| 11 | 4 mg BID | 165 | 50 | −70 | 1426 | 59 | stable |
| Melanoma | | | | | | | disease |
| 12 | 8 mg BID | 75 | 5 | −93 | ND | ND | |
| Breast | | | | | | | |
| 13 | 15 mg BID | 165 | 150 | −9 | 7680 | 320.0 | stable |
| Breast | | | | | | | disease |
| 14 | 15 mg BID | 20 | 0 | −100 | 2480 | 103.3 | |
| Melanoma | | | | | | | |
| 15 | 20 mg BID | 270 | 1 | −100 | 10560 | 440.0 | partial |
| Melanoma | | | | | | | response |
| 16 | 30 mg BID | 35 | 3 | −91 | 12000 | 500.0 | |
| Colon | | | | | | | |
| 17 | 30 mg BID | 73 | 70 | −4 | 12920 | 538.3 | stable |
| Melanoma | | | | | | | disease |
| 18 | 30 mg BID | 118 | 15 | −87 | ND | ND | |
| Melanoma | | | | | | | |
| 19 | 20 mg BID | 80 | 0 | −100 | ND | ND | |
| Melanoma | continuous | | | | | | |
| 20 | 20 mg BID | 80 | 23 | −72 | 5480 | 228.3 | stable |
| Melanoma | continuous | | | | | | disease |
| 21 | 20 mg BID | 55 | 45 | −18 | 5800 | 241.7 | |
| Melanoma | continuous | | | | | | |

*The data presented are limited to the subset of patients whose biopsies have been assayed for pERK activity.
The pERK values represent the average of two tissue sections on each tumor specimen.
ND—not determined.

We claim:

1. A dosage form for administration to a human suffering from abnormal cell growth in a body tissue, the dosage form comprising N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to reduce phosphorylation of ERK in the body tissue by at least 50%.

2. The dosage form of claim 1, wherein the phosphorylation of ERK is reduced by at least 90%.

3. The dosage form of claim 1, wherein the abnormal cell growth is cancer and ERK is cancer ERK.

4. The dosage form of claim 1, wherein a steady-state average plasma concentration value of N-[(R)-2,3-dihydroxy-propoxy]-3,4-difluoro-2-(2-fluoro-4-iodo-phenylamino)-benzamide or active metabolites thereof, is at least 7 ng/mL for at least 24 hours after administration to the human.

5. The dosage form of claim 1, wherein the dosage form comprises N-[(R)-2,3-dihydroxy-propoxy]-3,4-difluoro-2-(2-fluoro-4-iodo-phenylamino)-benzamide in an amount of from 1 to 30 mg.

6. The dosage form of claim 5, wherein the dosage form is an oral dosage form.

7. A method of treating cancer in a human, the method comprising administering to the human the dosage form of claim 3.

8. The method of claim 7, wherein the cancer is selected from mesothelioma, hepatobilliary (hepatic and billiary duct), a primary CNS tumor, a secondary CNS tumor, a primary brain tumor, a secondary brain tumor, NSCLC cancer, SCLC cancer, bone cancer, pancreatic cancer, skin cancer, head cancer, neck cancer, cutaneous melanoma, intraocular melanoma, ovarian cancer, colon cancer, rectal cancer, cancer of the anal region, stomach cancer, gastric cancer, colorectal cancer, duodenal cancer, breast cancer, uterine cancer, carcinoma of the fallopian tubes, carcinoma of the endometrium, carcinoma of the cervix, carcinoma of the vagina, carcinoma of the vulva, Hodgkin's disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney, cancer of the ureter, renal cell carcinoma, carcinoma of the renal pelvis, neoplasms of the central nervous system (CNS), primary CNS lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, and combinations thereof.

9. A dosage form for administration to a human suffering from abnormal cell growth in a body tissue, the dosage form comprising N-[(R)-2,3-dihydroxy-propoxy]-3,4-difluoro-2-(2-fluoro-4-iodo-phenylamino)-benzamide, a pharmaceutically acceptable salt or solvate, or a mixture thereof, in an amount effective to suppress a proliferation marker Ki67 in the body tissue by at least 30%.

10. The dosage form of claim 9, wherein the abnormal cell growth is cancer and Ki67 is cancer Ki67.

11. The dosage form of claim 9, wherein a steady-state average plasma concentration value of N-[(R)-2,3-dihydroxy-propoxy]-3,4-difluoro-2-(2-fluoro-4-iodo-phenylamino)-benzamide or active metabolites thereof, is at least 7 ng/mL for at least 24 hours after administration to the human.

12. The dosage form of claim 9, wherein the dosage form comprises N-[(R)-2,3-dihydroxy-propoxy]-3,4-difluoro-2-(2-fluoro-4-iodo-phenylamino)-benzamide in an amount of from 1 to 30 mg.

13. The dosage form of claim 12, wherein the dosage form is an oral dosage form.

14. A method of treating cancer in a human, the method comprising administering to the human the dosage form of claim 10.

15. The method of claim 14, wherein the cancer is selected from mesothelioma, hepatobilliary (hepatic and billiary duct), a primary CNS tumor, a secondary CNS tumor, a primary brain tumor, a secondary brain tumor, NSCLC cancer, SCLC cancer, bone cancer, pancreatic cancer, skin cancer, head cancer, neck cancer, cutaneous melanoma, intraocular melanoma, ovarian cancer, colon cancer, rectal cancer, cancer of the anal region, stomach cancer, gastric cancer, colorectal cancer, duodenal cancer, breast cancer, uterine cancer, carcinoma of the fallopian tubes, carcinoma of the endometrium, carcinoma of the cervix, carcinoma of the vagina, carcinoma of the vulva, Hodgkin's disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney, cancer of the ureter, renal cell carcinoma, carcinoma of the renal pelvis, neoplasms of the central nervous system (CNS), primary CNS lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, and combinations thereof.

16. A combination, consisting essentially of N-[4-[[(2,4-diamino-6-pteridinyl)methyl]methylamino]benzoyl]-L-glutamic acid, or a pharmaceutically acceptable salt thereof, and N-[(R)-2,3-dihydroxy-propoxy]-3,4-difluoro-2-(2-fluoro-4-iodo-phenylamino)-benzamide.
